(19) United States
(12) Patent Application Publication
(10) Pub. No.: US 2013/0150724 A1
(43) Pub. Date: Jun. 13, 2013

(54) BLOOD VESSEL ACCESS DEVICES, SYSTEMS, AND METHODS

(71) Applicant: Verathon Inc., Bothell, WA (US)

(72) Inventors: Michael Blaivas, Cumming, GA (US); Timothy Chinowsky, Seattle, WA (US)

(73) Assignee: Verathon Inc., Bothell, WA (US)

(21) Appl. No.: 13/763,210

(22) Filed: Feb. 8, 2013

Related U.S. Application Data
(63) Continuation of application No. 12/986,143, filed on Jan. 6, 2011, now abandoned.
(60) Provisional application No. 61/293,004, filed on Jan. 7, 2010.

(51) Int. Cl. A61B 8/08; A61B 8/00

(52) U.S. Cl. CPC A61B 8/085; A61B 8/461
USPC 600/461

(57) ABSTRACT

A device having a needle injector pivotally attached to an ultrasound transceiver is operated to place a sterilizable needle or needle/cannula unit within a blood vessel by a single user-device operator in which the blood vessel is made visible in a monitor image by ultrasound insonification. A guidance template is overlapped on at least one of a transverse, longitudinal, or three-dimensionally imaged blood vessel that illustrates a predicted path of the needle when it undergoes movement implemented by a controller. In alternate embodiments the needle injector, ultrasound transceiver, and needle or needle/cannula unit may be contained within a flexible sheath that is capable of being sterilized.

### CROSS REFERENCES TO RELATED APPLICATIONS

This application is a continuation of U.S. patent application Ser. No. 12/986,143 which claims the benefit of priority to and incorporates by reference in its entirety U.S. Provisional Patent Application Ser. No. 61/293,004 filed Jan. 7, 2010.

### FIELD OF THE INVENTION

Disclosure herein is generally directed to the field of blood vessel access related devices, systems, and methods.

### BACKGROUND OF THE INVENTION

Medical personnel can be faced with patients who present arteries or veins that are difficult to access with a needle and any needle-cannula assembly due to the qualities of the overlaying skin and/or the size and configuration of a given artery or vein, and the techniques undertaken to access a given blood vessel. The vein or artery may be obscured due to overlying fatty tissues or lack of sufficient blood flow may insufficiently fill the lumen to make the blood vessel palpable, as occurs with blown veins compromised with a hematoma, or veins that are otherwise structurally compromised as found in the elderly, intravenous administered drug users, and critically ill patients with very low blood pressure. Such patient as these, and with obese patients, proves difficult to cannulate under “blind” procedures. In many cases these patients have to endure multiple stabs with a needle, sometimes with penetration through the posterior wall of a vein before a successful placement of the needle is achieved and stable residence of the cannula or catheter within the blood vessel is achieved. Even allowing for an occasionally successful blind stick-and-insert catheter operation, the inserted catheter, if entered at too sharp an angle into a given blood vessel, may yet kink on insertion and thus hamper fluid delivery or removal into or from the blood vessel lumen. Moreover, current ultrasound image guided blood vessel access procedures require two people, one person to hold the ultrasound probe to secure an image to guide by, and another person to insert the needle/cannula. The prior art thus requires a minimum of three hands, a first person to hold the ultrasound transceiver and operate the ultrasound transceiver controls and nearby imaging systems, and a second person to handle and work in tandem in close proximity with the first person to handle and insert the needle/cannula while observing the ultrasound image procured from the first person. With current blood-access ultrasound image guided devices, the first person commonly utilizes both hands and second person at least one hand to do the needle insertion, for a minimum of three handed, and thus a two-person operation. With the disclosure detailed below, there are solutions for difficult-to-access blood vessels that do not require two people to perform ultrasound image-guided blood vessel access procedures.

### BRIEF DESCRIPTION OF THE DRAWINGS

Preferred and alternative examples of the present invention are described in detail below with reference to the following drawings depicted in FIGS. 1-44:

FIG. 1 schematically depicts a blood vessel access device that images blood vessels utilizing B-mode based single scan planes and rotationally-configured scan plane arrays;

FIG. 2A-2E schematically depict video image types generated by the blood access device;

FIG. 3 schematically depicts a blood vessel access device that images blood vessels utilizing A-mode based 3D-distributed scan lines;

FIG. 4 schematically depicts a blood vessel access device that images blood vessels utilizing B-mode single scan planes, fan-configured B-mode scan plane arrays, and C-mode scan planes;

FIG. 5 schematically depicts peripheral blood vessels located in a patients forearm accessible by the blood vessel access devices of depicted in FIGS. 1, 3, 4, 6; and 18-26;

FIG. 6 schematically depicts another embodiment of a blood access device;

FIG. 7 schematically depicts click-based needle positioning and catheter advance controls configurable for the blood access devices depicted in FIGS. 1, 3, 4, 6; and 18-26;

FIG. 8 schematically depicts click-based ultrasound transducer controls configurable for the blood access devices depicted in FIGS. 1, 3, 4, 6, and 18-26;

FIG. 9 schematically depicts a 2D transverse cross-sectional sonogram that courses substantially perpendicular to the long axis of the vein;

FIG. 10 schematically depicts a 2D longitudinal cross-sectional sonogram that courses substantially parallel to the long axis of the vein;

FIG. 11 schematically depicts a 3D cross-sectional sonogram of the vein reconstructed from multiple scan planes rotationally positioned across the vein;

FIG. 12 schematically depicts a guidance template applicable to a longitudinal cross-sectional venous sonogram;

FIG. 13 schematically depicts a guidance template applicable to a transverse cross-sectional venous sonogram;

FIG. 14 schematically depicts the guidance template overlaid on the longitudinal cross-sectional venous sonogram;

FIG. 15 schematically depicts the guidance template overlaid on the transverse cross-sectional venous sonogram;

FIG. 16 depicts a system block diagram of the components relating to the arm, transceiver probe, and console of particular embodiments utilizing the device described herein;

FIG. 16A depicts a system block diagram relating to the blood vessel access device depicted in FIG. 18 having injector controls located within the injector arm;

FIG. 17 schematically depicts a configuration of injector arm components to advance or retract needle or needle/cannula assemblies;

FIGS. 18-26 schematically depict another embodiment of a blood access device;

FIGS. 27-29 schematically depict needle pathway prediction plots;

FIGS. 30-36 depict different ultrasound image area presentations on the touch sensitive monitor 124 positioned by engagement of image position buttons 130, 132, 134, and 136.

FIGS. 36-37C depict sterile sheath blood vessel access device embodiments and their operation;

FIGS. 38-40 schematically depict cable and wireless communicated blood vessel access device and system; and

FIGS. 41-44 schematically depict components of the cart-deployed cable based blood vessel access device and systems.

### DETAILED DESCRIPTION OF THE INVENTION

The invention concerns a single-person operable device configured for projecting ultrasound energy into a patient and generating an ultrasound image that may be used to guide a needle and a catheter or cannula under precise mechanical control and place the catheter or cannula reliably into the patient's vascular structure. The device is configured to allow the single person user-operator to perform both the acquisition of ultrasound images used for ultrasound image-guided blood vessel access procedures and to implement needle and catheter/cannula placement within the imaged, targeted blood vessel with either the device user's single hand or two hands.

The embodiments include an ultrasound transceiver that is pivotally attached to a needle injector and operated to place a sterilizable needle or needle/cannula unit within a specifically targeted blood vessel made visible in a real-time monitor image by ultrasound insonification. A guidance template is overlapped on at least one of a transverse, longitudinal, or three-dimensionally imaged blood vessel that illustrates a predicted path of the needle when it undergoes movement implemented by a needle-and-cannula motion controller manipulated in a one-handed user operation. In alternate embodiments the needle injector, ultrasound transceiver, and needle or needle/cannula unit may be contained within a flexible sheath that is capable of being sterilized and has a sonic gel pouch that can be opened to release its contents to make sonic connection between the patient, the sterilized bag, and the ultrasound transducer. Signal connection to a monitor to provide ultrasound images for image guided insertion of needles or needle/cannula assemblies may be from the ultrasound transducer that are either cabled or wirelessly connected to present the real-time images to a nearby monitor. Image processing and display allow for real time targeting of a blood vessel for needle penetration and a catheter or a cannula insertion, and to ascertain whether the targeted blood vessel is an artery or a vein before needle and subsequent cannulation procedures are implemented. The needle insertion and cannulation is undertaken in a single-user, one-handed operation or a two-handed operation.

In greater detail, these embodiments relate to blood vessel access systems, devices, and methods for placing a needle within the lumen of at least one blood vessel. The blood vessel access devices aid the user in insertion of peripheral intravenous (IV) lines, central, and peripherally inserted central catheter PICC lines by improving both the visualization of the vasculature and manipulation of the needle. A compact ultrasound probe located in a transceiver handset provides real-time B-mode images of the anatomy to be cannulated. A motorized mechanism contained in an injector arm attached to the probe advances the needle and catheter into the ultrasound visualized blood vessel under local control from the user. As regards systems, disclosure illustrated and discussed below are drawn to an ultrasound transceiver that is sonically coupled to convey ultrasound energy into a patient, and to generate at signals from received returning ultrasound echoes to generate at least one image of the patient's sonicated region on a monitor in which the at least one image includes a single or multiple blood vessels are ultrasonically made visible within the real time image. The system further includes a needle injection that is pivotally attached or connected with the ultrasound transceiver. The needle may be attached to an overlapping cannula, and the needle and/or overlapping cannula may be contained within a sterilizable housing that is detachably connectable with the needle injector. The needle injector is connected with a controller that controls the advancement or retraction from of the needle from the sterilizable housing. The system further includes software or executable programs having instructions configured to develop and overlay at least one aiming template or guidance template. The aiming or guidance overlay includes a predicted path that the needle will undertake to reach and penetrate the lumen of the at least one blood vessel. The guidance overlay includes the predicted path to be undertaken on at least one of a transverse or lateral cross-sectional view, a longitudinal cross-sectional view, and a three dimensional view of the at least one blood vessel presentable within the at least one image.

In other embodiments the access system, including the ultrasound transceiver, the injector, and any detachable needle/cannula housing units, may be enveloped within a flexible sheath that is capable of being sterilized. Sonic coupling gel may be applied between the transceiver and the internal surfaces of the flexible sheath, and between the patient and the external surface of the flexible sheath.

As regards to an access device for purposes of executing the image guided placement of a needle within at least one blood vessel, the access device includes pivotally connecting the access device to an ultrasound system. The ultrasound system includes a monitor and may be portable to assist in obtaining images of blood vessel beneath the neck, chest, abdomen, arms, legs, and other part of the torso that is ultrasonically visualizable. As with the access system, the access device includes software or executable programs configured to develop and overlay aiming or guidance templates of predicted needle pathways onto at least one of a transverse cross-sectional view, a longitudinal cross-sectional view, and a three dimensional view of the at least one blood vessel presentable within the at least one image.

Similarly in other embodiments, the access device and pivotally connected ultrasound transceiver, including any detachable needle/cannula housing units, may be enveloped within a flexible sheath that is capable of being sterilized. Sonic coupling gel may be applied between the transceiver and the internal surfaces of the flexible sheath, and between the patient and the external surface of the flexible sheath.

As regards methods of using an access device or access system, the method encompasses connecting a needle injector pivotally with an ultrasound transceiver having a monitor configured to present an image of at least one blood vessel, installing a sterilizable housing containing the needle and cannula, and operating the needle injector controller to place the needle within the lumen of at least one blood vessel presented on the monitor to which is overlaid a guidance template.

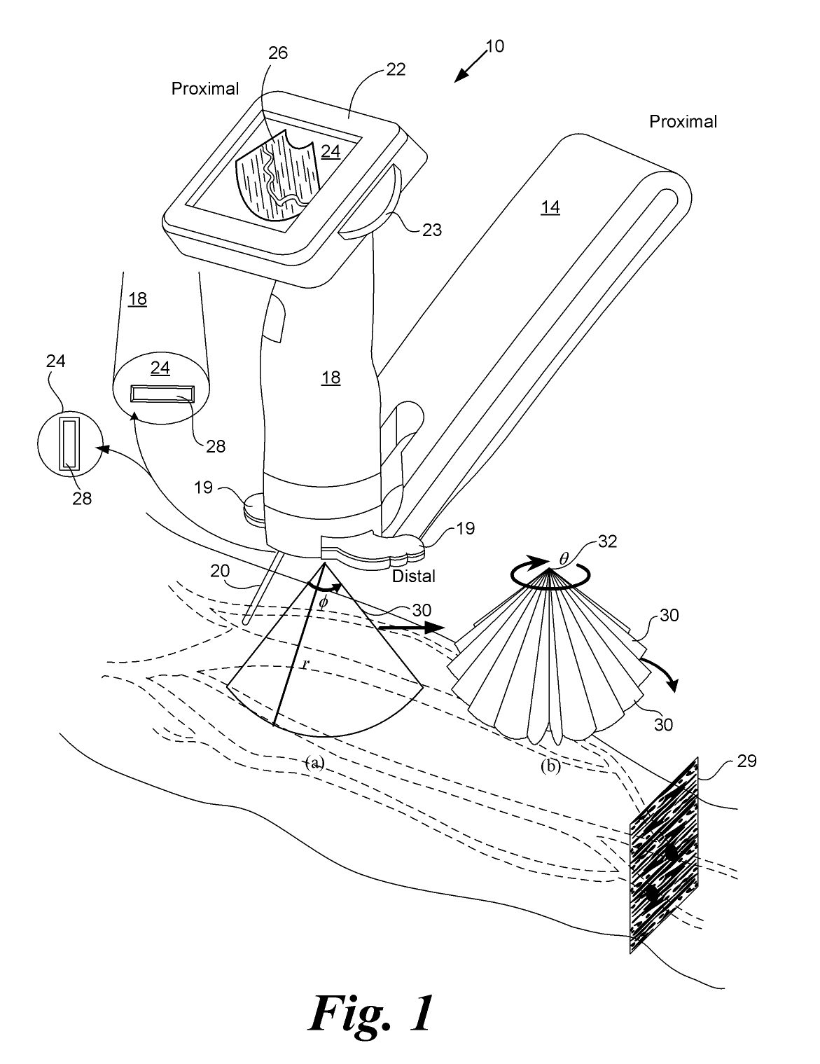

Different embodiments of blood vessel access devices, systems, and method of using devices and systems are described in FIGS. 1-44 below. The devices, systems, and methods may be employed to target any blood vessel to allow hospital or clinic based personnel to undertake successful ultrasound-guided placement of short peripheral intravenous solutions (IVs), generally under aseptic conditions, and peripherally inserted central catheter (PICC) lines, and any difficult medical procedure currently using blind needle placement, generally under sterile conditions. Difficult medical procedures include nerve blocks, Thoracentesis and Paracentesis procedures, and biopsy procedures. Needles utilized by the devices and systems commonly cover 22 to 16 gauge needles and with the appropriate larger sized cannula or catheters that may be slidable over the 22 to 16 gauge needles.

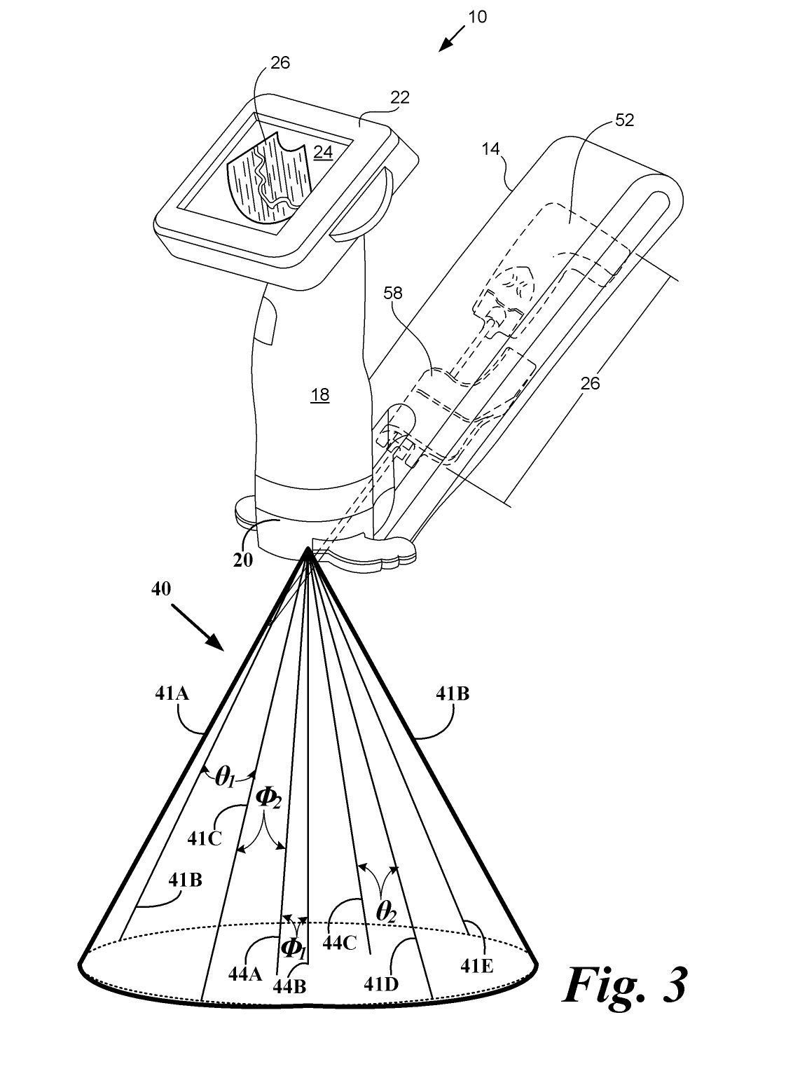

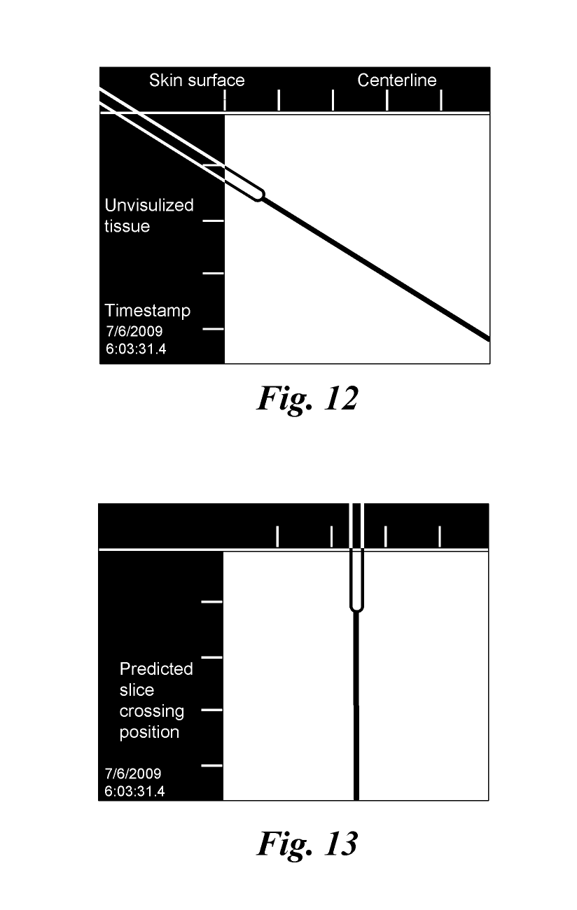

FIG. 1 schematically depicts a blood vessel access device 10 that images blood vessels utilizing B-mode based single scan planes 30 and rotationally-configured scan plane array 32 derived from multiple scan planes 30 rotationally pivotable about its apex separated by an angle theta θ. Each scan plan 30 is derived from multiple A-mode scan lines having radius r that rotationally pivots about its apex separated by angle phi φ. The blood vessel access device 10 utilizes an injector arm 14 having a proximal and a distal end. The injector arm 14 houses an injector apparatus 50 illustrated in FIGS. 3 and 17 below. Shown in the insets is a distal end 24 of transceiver 18 that houses an ultrasound transducer 28 adjacent to the distal end of injector arm 14. The transducer 28 is rotatable to obtain a two dimensional (2D) transectional cross-sectional view, a 2D longitudinal cross-sectional view, or a three dimensional (3D) view of blood vessels. The injector arm 14 is pivotally attached to an ultrasound transceiver 18 that similarly includes a distal and proximal end. Friction hinges 19 located in the distal portion of transceiver 18 provide for securely adjusting and holding to a user-selected angular arrangement of the injector arm 14 relative to the long axis of the transceiver 18. Friction hinges 19 allows the user to change the angle of the injector arm 14 with respect to the transducer 28 surface. The friction hinge 19 is substantially stiff enough to keep the angle constant unless the injector art 14 is intentionally moved or pivoted by the user. Inside the injector arm resides an angle sensor (not shown) that continually measures the angle of the injector arm 14 relative to the transducer 28. The angular data obtained is used to construct the on-screen displays showing the predicted or needle trajectory or trajectories as described in FIGS. 12-15 and 27-28. Located at the proximal end of the transceiver 18 is a monitor 22 housing image adjustment control 23 and monitor screen 24. A needle 20 protruding from the distal end of the injector arm 14 may intersect a given B-mode ultrasound scan plane 30 and thus become visible to the user in the monitor screen 24. Presented on screen 24 is a 2D longitudinal cross-sectional view 26 of a vein viewed from the patients mid forearm. Similarly, were the transducer 28 be sonically coupled or pressed against near the wrist end of the forearm a transactional cross-sectional view 29 would be similarly presentable on the screen 24.

The ultrasound transducer 28 may also be configured to obtain 2D and 3D images of blood vessels, either arteries and/or veins, located in peripheral appendages such as the arm or leg, but also in the trunk and neck. As illustrated below, the ultrasound imaging may exploit A-mode, B-mode, and C-mode ultrasound configurations. In one alternate embodiment, the ultrasound transducer 28 may be comprised of a 128 element linear array transducer configured to emit and receive ultrasound in the 7-10 MHz frequency range, and any harmonics thereof. B-mode imaging may commonly visualize 25 mm×40 mm tissues slices. As shown in the inset above, the transducer 28 may be rotated to get a complete ultrasound image of the puncture site. Furthermore, Doppler based ultrasound may be employed to distinguish artery blood vessels from vein blood vessels.

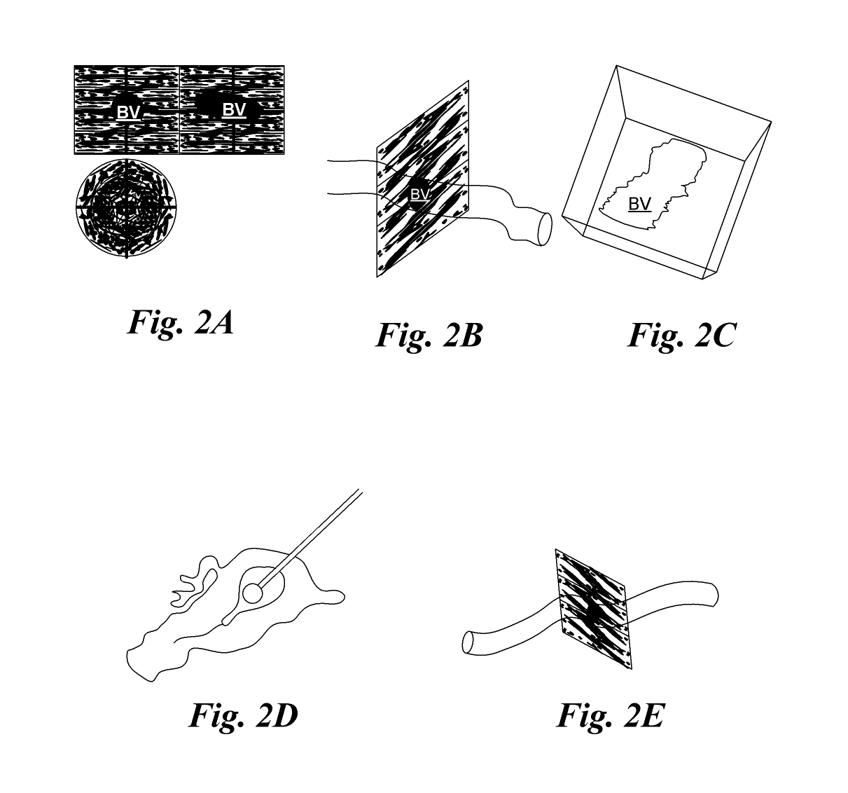

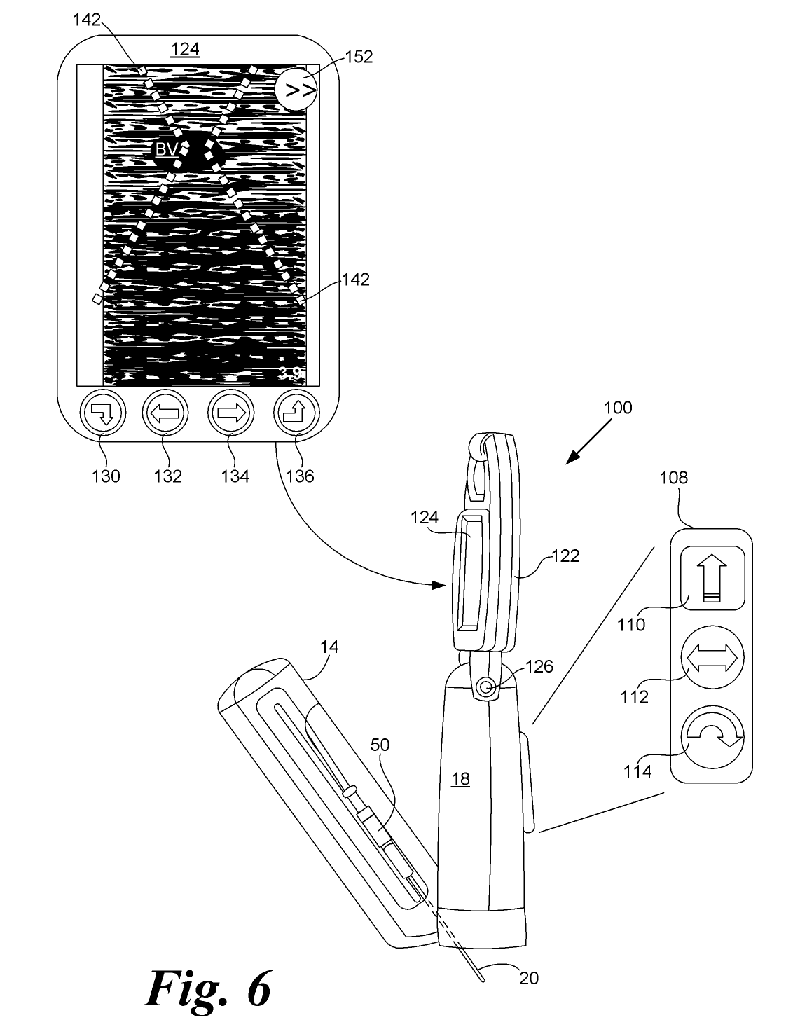

FIG. 2A-2E schematically depict video image types generated by the blood access device 10 depicted in FIGS. 1, 3, and 4 and blood access devices 100, 200, 500, 600, and 700 respectively depicted in FIGS. 6, 18, 41, 42 and 43.

FIG. 2A schematically illustrates 2D transactional of several peripheral veins.

FIG. 2B schematically illustrates a 3D visualization of peripheral veins.

FIG. 2C schematically illustrates a 3D detection and reconstruction of a peripheral vein.

FIG. 2D schematically illustrates a 3D visualization of a vein complex located in a forearm of a patient.

FIG. 2E schematically illustrates a 3D detection and reconstruction of a peripheral vein along the longitudinal axis of the peripheral vein. The 3D images conveyed may include plots that combine live ultrasound images with pint clouds generated from the other scan planes.

FIG. 3 schematically depicts the blood vessel access device 10 that is configured to ultrasonically image blood vessels utilizing A-mode based 3D-distributed scan lines. In this configuration a scan cone 40 emanates from the transducer 28 and included peripheral scan lines 41A/B/C/D and internal scan lines 44A/B/C/D. Computer executable instructions provide for 2D cross sections and 3D image reconstructions similar to those presented in FIGS. 2A-2E from anatomical and needle structures that are captured within the scan cone 40. Coordinates for reconstruction of images utilize the length of a given scan line from the apex of the scan cone 40, and angular values between the scan lines. The angular values include angle theta one (θ₁) that provides the angular separation between any two peripheral scan lines 41, angle phi one (φ₁) that provides the angular separation between any two internal scan lines 44, and angle phi two (φ₂) that provides the angular separation between any peripheral scan line 41 and internal scan line 44.

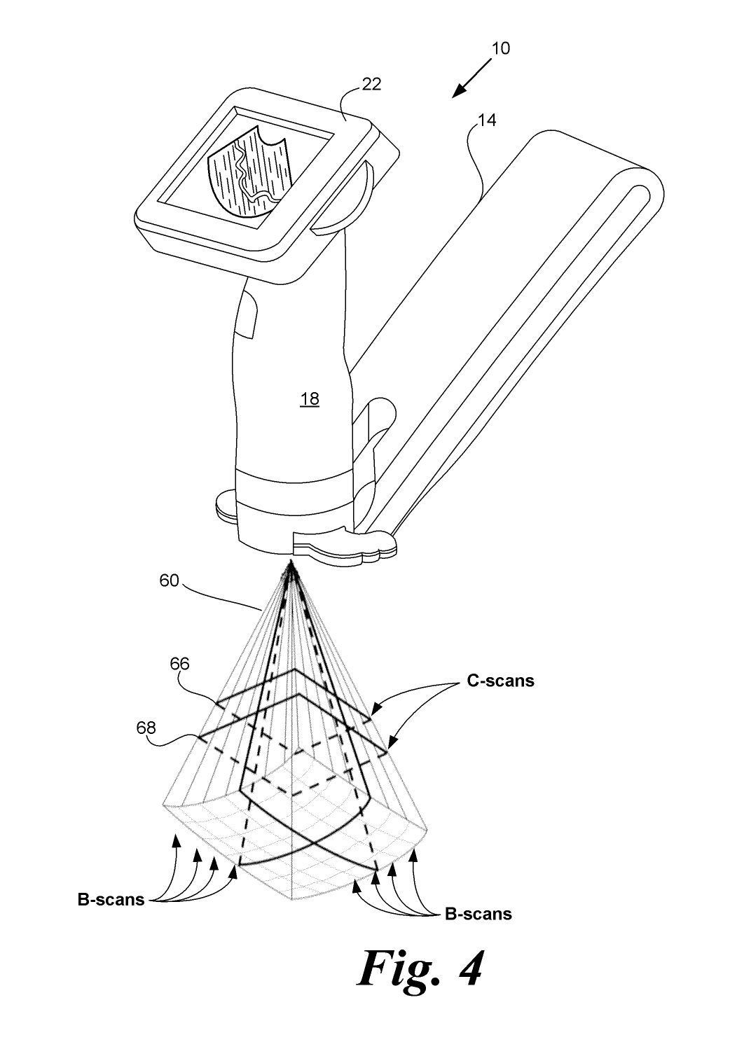

FIG. 4 schematically depicts the blood vessel access device 10 that is configured images blood vessels utilizing B-mode single scan planes, fan-configured B-mode scan plane arrays, and C-mode scan planes. Transection cross sections, longitudinal cross-sections, and 3D images may be obtained from fan-configured B-mode scan plane arrays and depth based C-mode scan planes. Scan cone 60 is shown emanating from the ultrasound transducer 28 and composed of a series of B-scan planes arranged in fan-like configuration about the apex. One set of B-scan planes can be seen perpendicular to the other set of B-scan planes. C-scan planes 68 are shown at different depths from the apex of the scan cone 60.

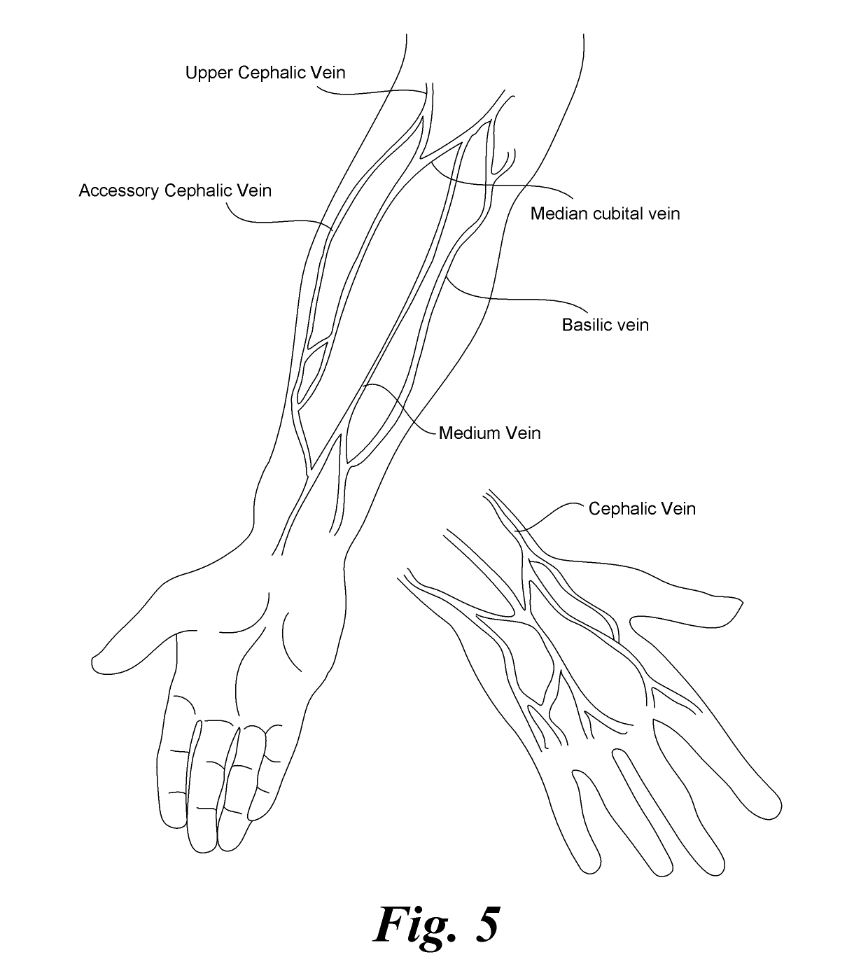

FIG. 5 schematically depicts peripheral blood vessels located in a patients forearm accessible by the blood vessel access devices of depicted in FIGS. 1, 3, 4, 6; and 18-26. The upper cephalic vein lies above the antecubital space and can be difficult to visualize and stabilized. Using 22 to 16-gauge catheters, this blood vessel is often suited for midline catheter or PICC lines. The accessory cephalic vein is located on the top of the forearm, fairly easy to see and stabilize, and can accommodate 22 to 18-gauge catheters. In general, the catheter 21 tip should not b placed in the bend of the arm. The median vein originates in the palm of the hand and can accommodate 24- to 20-gauge catheters 21. The basilica vein, though large and easy to see, tends to roll and is difficult to stabilize. Nonetheless, it can accommodate 22- to 16-gauge catheters, especially when the patient's arm is placed across the patient's chest and the blood vessel access device user is standing opposite the side of the bed to perform the venipuncture operation. The cephalic vein can accommodate 22- to 16-gauge catheters and is suitable for infusing chemically irritating solutions and blood products. To avoid the nearby radial nerve, venipuncture operations using the blood vessel access devices described herein are implemented 10 to 12.5 cm above the level of the wrist and not in the wrist.

FIG. 6 schematically depicts another embodiment of a blood vessel access device 100. Similar configured to the device 10, blood vessel access device 10 includes a monitor head 122 that may be rotated or adjusted for optimum viewing of the monitor screen 124 by the user via pivots 126. The tilting of the display head 122 accommodates the user when sitting or standing. Monitor screen 124 provides a touch screen user interface to permit the user to adjust the appearance of the ultrasound images and/or call up alphanumeric information or to engage other portions of the monitor screen to ascertain whether the targeted blood vessel BV is a vein or an artery. The ultrasound transceiver handle and display head 122 can be rotated relative to the injector arm 14 to accommodate right or left hand use. The pivots 124 assist in reducing inadvertent user motion. Within monitor display 124 are targeting lines 142 each having a bend or apex within the blood vessel BV shown here in transaction. The inflection point of the targeting lines indicates the depth beneath the skin's surface or from the ultrasound transducer 28 the blood vessel BV resides.

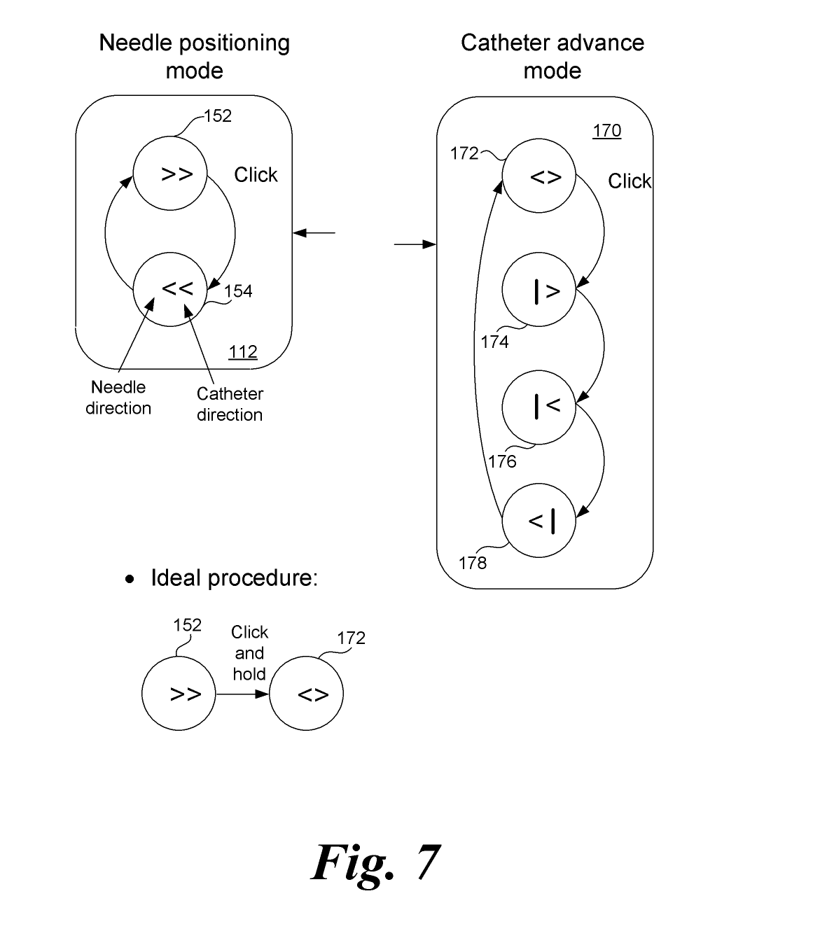

On the ultrasound transceiver handset 18 are ultrasound transducer and injector motion controls 108. Motion controls 108 may include needle motion button 110, needle motion direction button 112, and ultrasound transducer positioning button 114. The needle motion button 110 is configured to advance the needle to penetrate or retract the needle 20 from the patient depending on the engagement of the needle direction button 112. The needle advance control 110 is a force sensitive button that controls the injector mechanism 50 speed. When pressed harder the needle advance button 110 acts as an accelerator in that the injector mechanism 50 moves faster. The motion controls 108 also include needle direction control 112. The needle direction control 112 functions as a “gear shift” from forward (needle penetration) to reverse (needle withdrawal or retraction). The speed of the needle penetration or withdrawal is governed by the force-sensitive needle motion button 110. Transducer position button 114 changes the orientation of the transducer 128 from a lateral position to a longitudinal position. Operation of the motion controls 108 provide for one-handed user manipulation to position a needle 20 or needle/cannula within an ultrasound viewable image. Double click operation icon 152 described in FIG. 7 below appears to indicate readiness to advance needle 20 into the blood vessel BV. Positioning or centering of the BV within the screen 124 is determined by position controls down 130, left 132, right 134, and up 136.

FIG. 7 schematically depicts click-based needle positioning and catheter or cannula advancement modes configurable for the blood access devices depicted in FIGS. 1, 3, 4, 6; and 18-26 allowing needle 20 and cannula placement within a targeted blood vessel by a user employing a single handed operation. Operation of these controls effect the movement direction of the needle slider 52 and cannula slider 56 of injector 50 described in FIG. 17 below. One button needs to select between many different combinations of needle and catheter/canula movement and advantageously provides to the user a balance of ease of use with versatility.

Under the needle positioning mode, double right arrow click operation 152 of needle direction button 112 advances the needle 20 via and left double click operation 154 selects the catheter or cannula movement direction. Double click operation 152 is indicated on the button overlay illustrated on monitor 124 of FIG. 6. Catheter or cannula advancement mode 170 describes click operations 172, 174, 176, and 178. Clicking button 172 selects the advancement or withdrawal of the cannula. Clicking button 174 increases the speed for advancement or delivery into the cannula into a targeted blood vessel BV by speed increments of one. Clicking button 176 increases the speed for withdrawal or removal of the cannula from the targeted blood vessel BV by speed increments of one. In an idealized or optimal procedure, clicking and holding button 152 engages the cannula direction button 172.

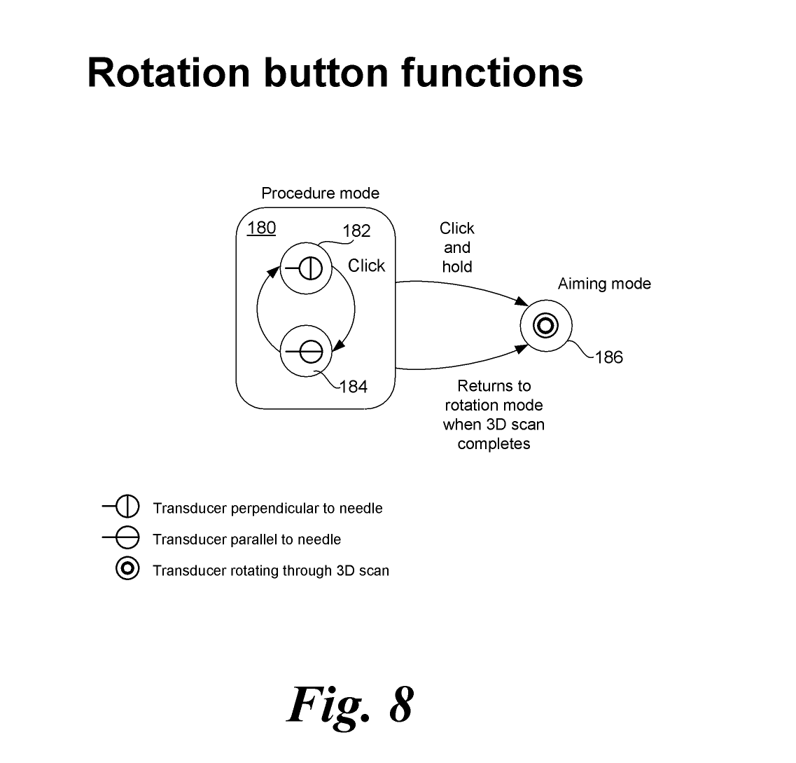

FIG. 8 schematically depicts click-based ultrasound transducer control 114 configurable for the blood access devices depicted in FIGS. 1, 3, 4, 6, and 18-26. In Procedure mode 180, clicking of button 182 orientates the transducer 128 substantially perpendicular to the needle 20 BV. Clicking of button 184 orientates the transducer 128 substantially parallel to the needle 20 BV. Whenever clicking and holding either button 182 or 184 engages the aiming mode wherein button 186 orientates the transducer 128 into rotation mode to obtain a 3D scan.

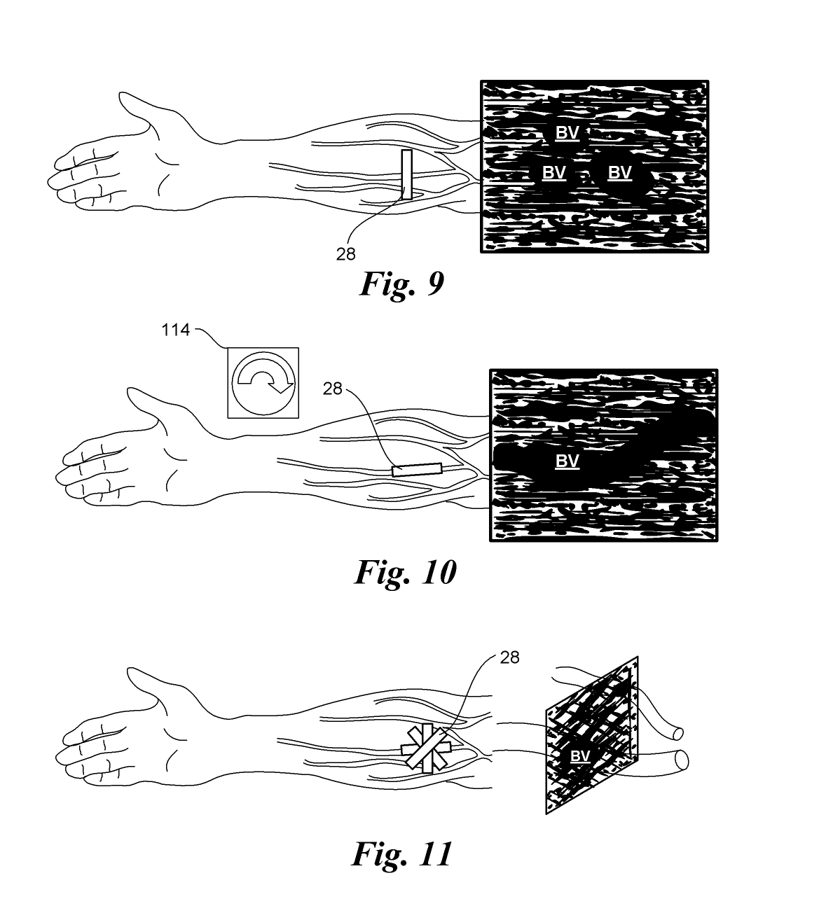

FIG. 9 schematically depicts a 2D transverse or lateral cross-sectional sonogram that courses substantially perpendicular to the long axis of the vein. Dark, circular areas represent blood vessels BV and may be artery or veins. The size of the blood vessel's BV lumen is visualized and its distance from the transducer or depth beneath the skin is ascertainable from alphanumeric data presentable on the sonograms 26 or 29 depicted in FIG. 1. In the image depicted in FIG. 9, the size of the blood vessel's BV lumen is at a lateral orientation as the ultrasound transducer 28 has a substantially perpendicular orientation to the long axis of the blood vessel BV.

FIG. 10 schematically depicts a 2D longitudinal cross-sectional sonogram that courses substantially parallel to the long axis of the blood vessel BV. Dark, sinuous columns or tunnels are ultrasonically visualized the blood vessel's BV lumen at a substantially parallel orientation to the long axis of the blood vessel. The change in orientation of the transducer 28 from a substantially perpendicular to a substantially parallel position is effected by the transducer motion controller 114 described in FIG. 6.

FIG. 11 schematically depicts a 3D cross-sectional sonogram of the vein reconstructed from multiple scan planes rotationally positioned across the blood vessel BV. Image reconstruction provides a bas-relief presentation.

FIG. 12 schematically depicts a guidance template applicable to a longitudinal cross-sectional venous sonogram. The guidance template illustrates the predicted route of the needle 20, including the un-visualized tissue and the visualized tissue that will occupy the white box.

FIG. 13 schematically depicts a guidance template applicable to a transverse cross-sectional venous sonogram. The guidance template illustrates the predicted route of the needle 20, including the un-visualized tissue and the visualized tissue that will occupy the white box.

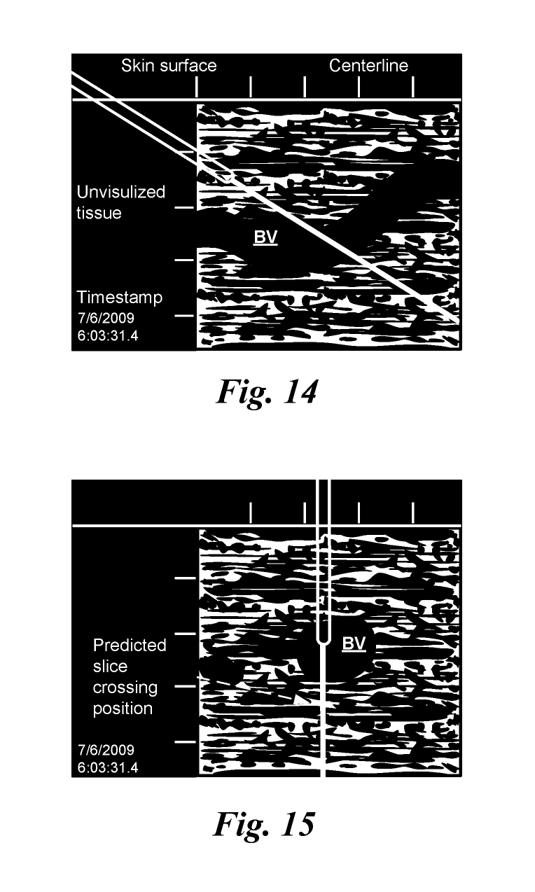

FIG. 14 schematically depicts the guidance template overlaid on the longitudinal cross-sectional venous sonogram. The predicted entry path also includes the route that will penetrate the distal wall of the blood vessel BV if the needle 20 is advanced too far.

FIG. 15 schematically depicts the guidance template overlaid on the transverse cross-sectional venous sonogram. The predicted entry path also includes the route that will penetrate the distal wall of the blood vessel BV if the needle 20 is advanced too far.

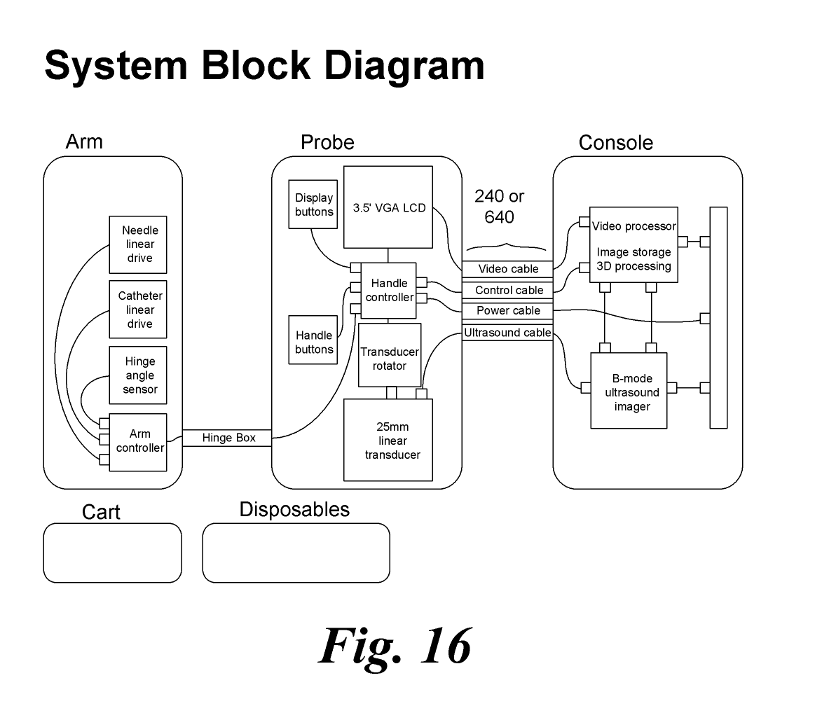

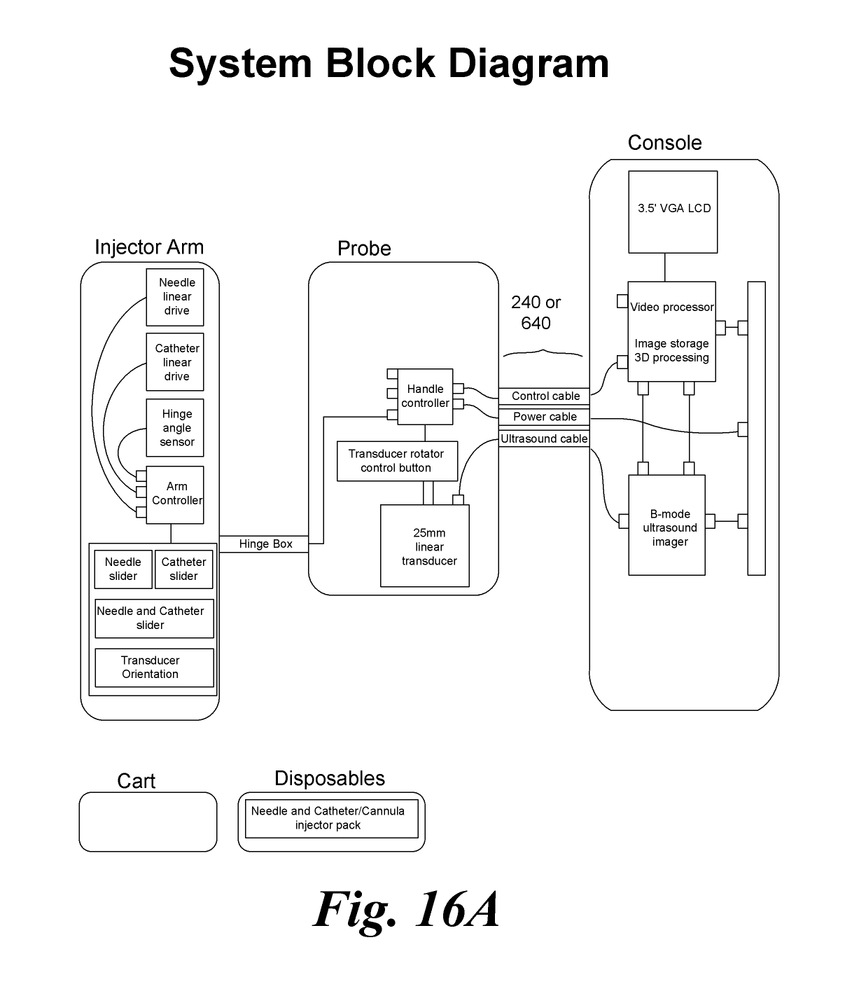

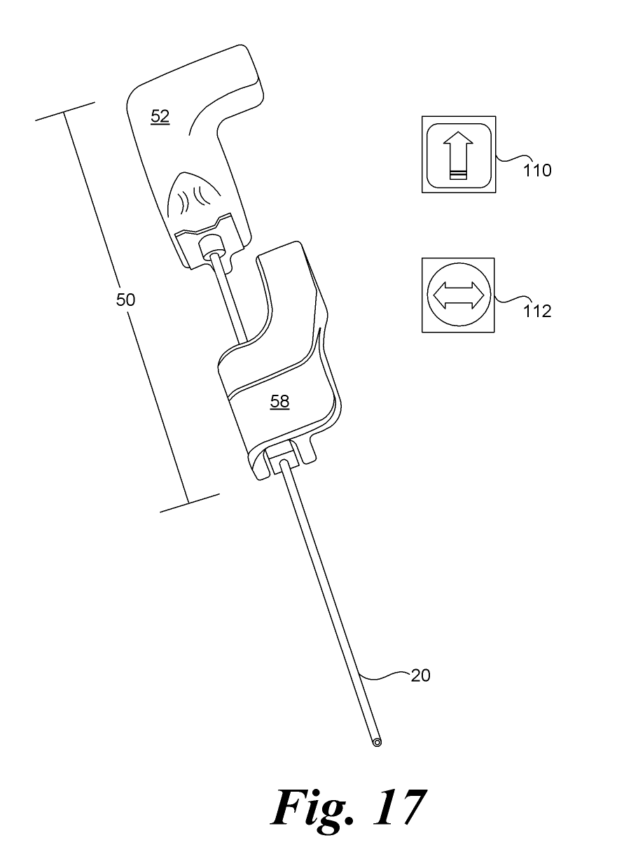

FIG. 16 schematically depicts a system block diagram. The system block diagram includes an Arm section, a Probe section, a Console section, a Cart section, and a Disposables section. The arm section concerns injector arms 14 and 214 and includes an arm controller block, a hinge angle sensor block, a catheter linear drive block, and a needle linear drive block. The probe section concerns ultrasound handle transceivers 18 and 218 and includes a display button block, a display screen block denoted as a 3.5 VGA LCD, a handle controller, handle buttons, transducer rotator block, and linear transducer block.

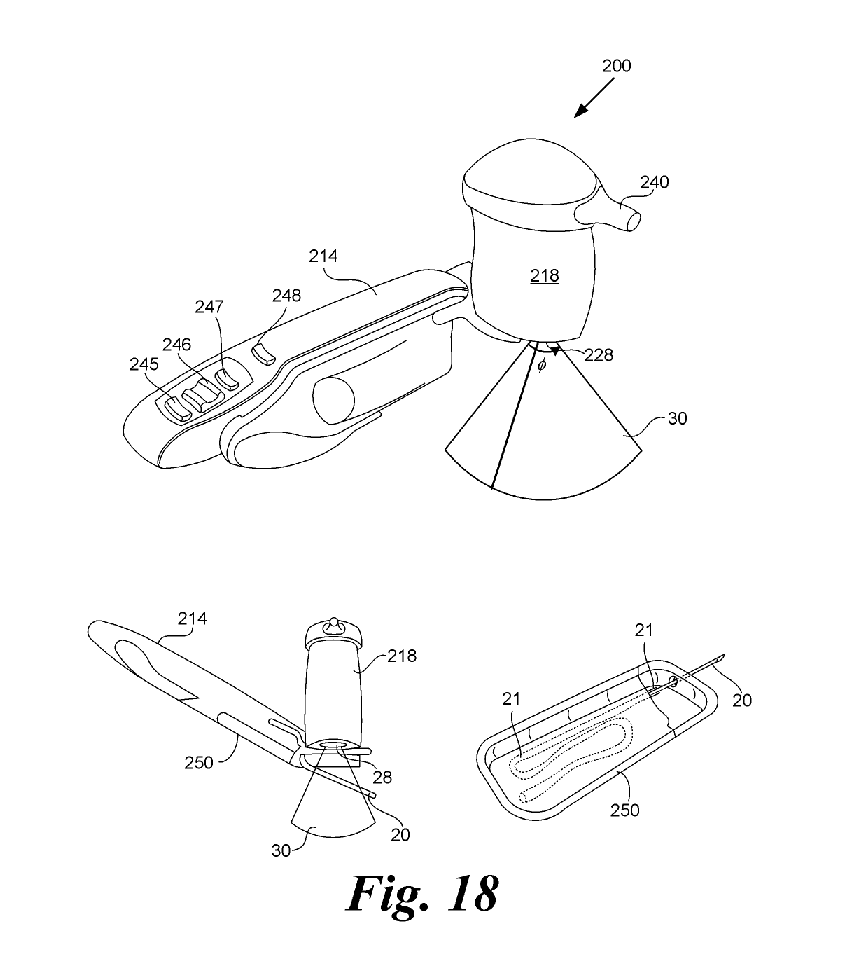

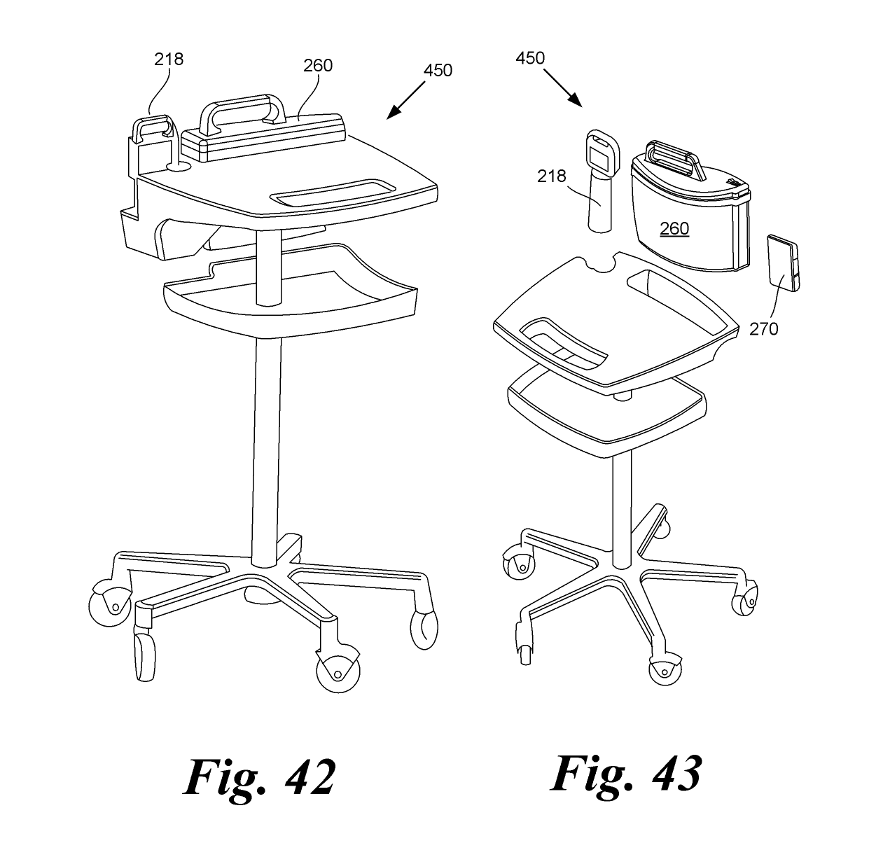

The Console section concerns portable console 260 described in FIGS. 41-43 with regards to transducer and communications connection with portable blood access devices 200 and 500 described in FIGS. 18-26 and 41 or the console equivalents built into the portable blood access devices 10, 100, 600 and 700 described in FIGS. 1, 3, 4, 6, and 42-43. The Console section includes a video processor block, an image storage 3D processing block, and a B-mode ultrasound imager block. Other blocks (not shown) may include A-mode block and C-mode ultrasound block. Between the Probe and Console sections includes transducer and communication cable component 240 described in FIG. 18 or its equivalent wireless transducer and communication connection 640 described in FIGS. 42-43. Hinge Box represents the mechanical connection between the injector arm and the transceiver probe sections. The cable 240/wireless connection 640 component provides for a video cable component, a control cable component, a power cable component and an ultrasound cable component.

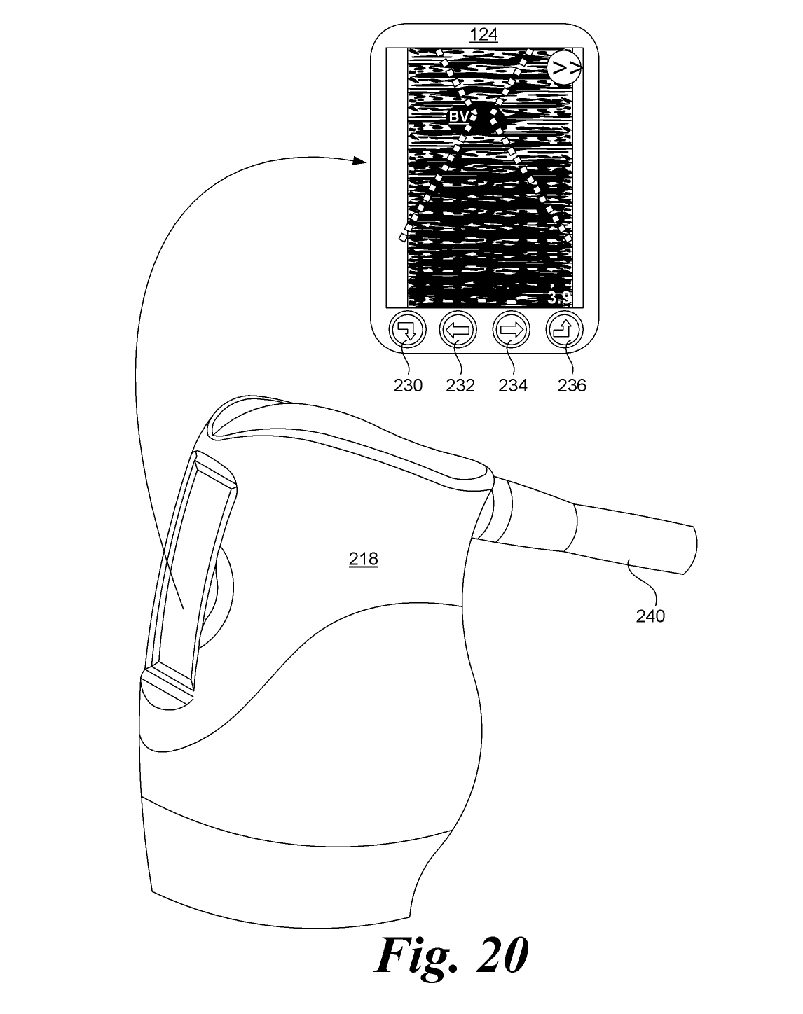

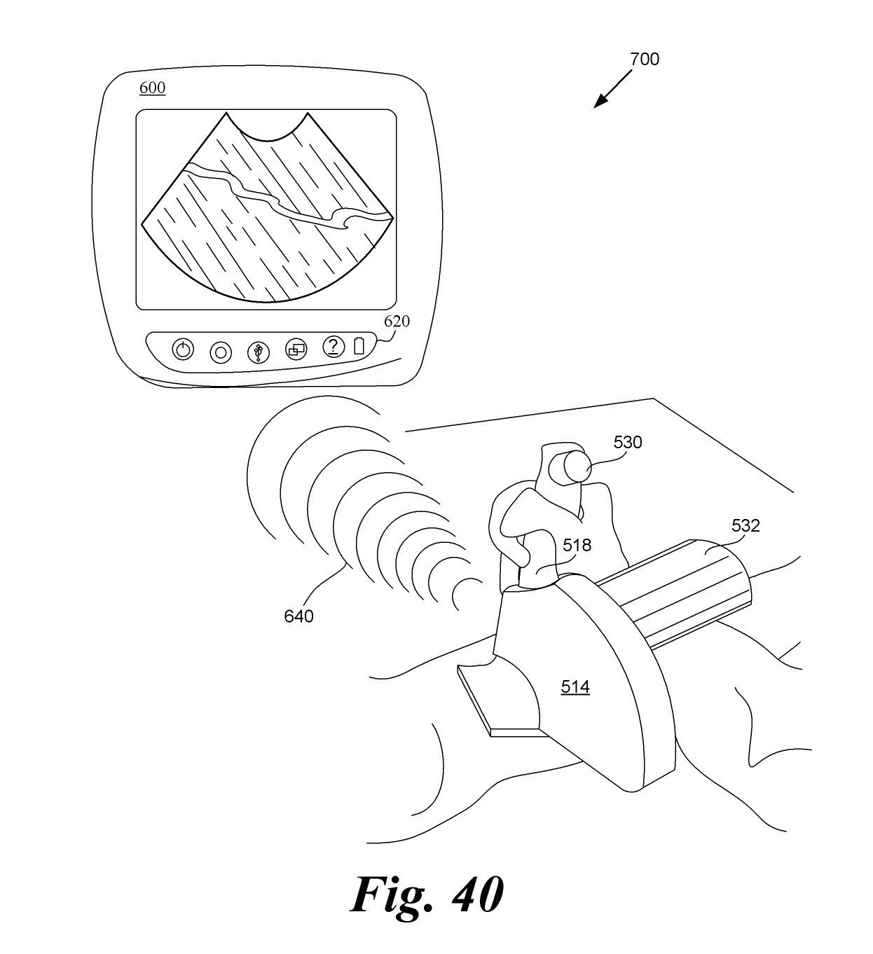

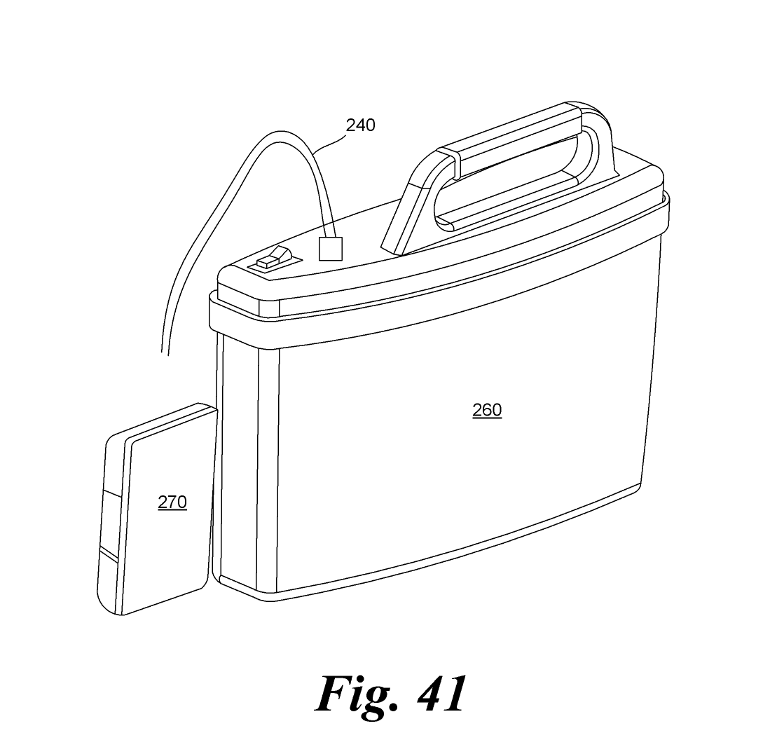

FIG. 16A depicts a system block diagram relating to the blood vessel access device depicted in FIG. 18 having injector controls located within the injector arm and touch sensitive image adjustment controls built into a touch sensitive screen further described in FIG. 20 below. Having many of the similar system block layouts as FIG. 16, the components within each injector section, ultrasound transceiver probe section, and console section are re-arranged from the system block diagram of FIG. 16 above. FIG. 16A calls out the blood vessel access device described in FIGS. 18-26 below. Included in the arrangement of mechanical functions the arm controller component controls and details concerning the disposables section. As regards mechanical functions, and with reference to FIG. 18 below, the arm controller component controls individually the needle slider to advance or retract the needle 20, the catheter slider to advance or retract the catheter/cannula 21, the needle and catheter slider which is controlled by the rocking switch 246 depicted in FIG. 18, and the transducer orientation that causes the repositioning of the ultrasound transducer to be oscillate from substantially a lateral view of a targeted blood vessel BV to a substantially longitudinal view of the targeted blood vessel BV. The ultrasound transceiver probe section includes the handle controller component, the transducer rotator control button, and the linear transducer, her denoted as a 25 mm linear transducer. The console section includes the touch screen monitor with built-in touch sensitive image control icons, here denoted as a 3.5 inch Video Graphics Array (VGA) Liquid Crystal Display (LCD). The ultrasound cable conveys signals between the linear transducer of the Probe section to the B-mode ultrasound imager of the Console section. In alternate embodiments the B-mode imager may be configured for C-mode and A-mode ultrasound. The disposable include the pre-sterilized injector pack 250 having the needle 20 and cannula/catheter 21. While control and communication connection to the portable console 261 depicted in FIG. 41 may utilize the wired cable 240 wherein the wired cable for this configuration includes a control cable component, a power cable component, and an ultrasound cable component. Similarly, a wireless equivalent signal 640 depicted in FIG. 40 may be configured for blood vessel access device 200 depicted in FIG. 18.

FIG. 17 schematically depicts a configuration of injector arm components to advance or retract needle or needle/cannula assemblies. Needle injector 50 includes a needle motion slider 52 and a cannula/catheter motion slider 56. The needle motion slider 52 grasps the shaft of the needle 20 in a position aligned with the ultrasound probe transducer 28. Grasping by the shaft reduces positional route prediction error due to needle bending during insertion. Upon receiving motion advancement signals the needle motion slider advances the needle 20 to penetrate the patients arm and into the ultrasound visualized blood vessel in reference to the guidance templates discussed above. Viewing a longitudinal cross section ultrasound image, and upon receiving motion advancement signals, the cannula slider 56 is advanced to slide the larger gauge cannula over the smaller gauge needle 20 now residing within the patient's artery or vein. When the cannula is visually observed to be residing within the patient's targeted blood vessel, and upon receiving motion withdrawal signals, the needle slider 52 is retracted, and the needle 20 pulled out of the cannula residing in the targeted blood vessel.

FIGS. 18-26 schematically depict another embodiment of a blood access device.

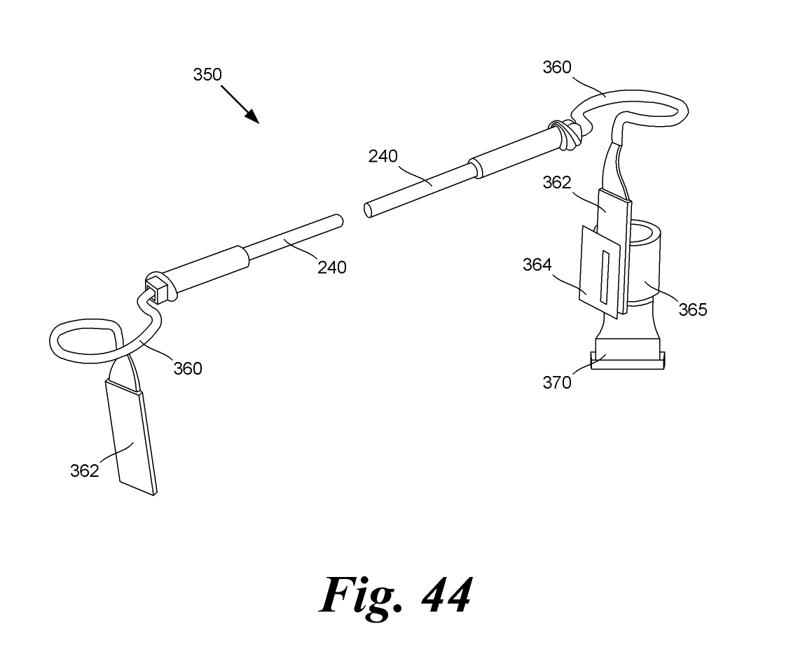

FIG. 18 schematically depicts a blood vessel access device 200 having an injector arm pivotally attached via a friction hinge (not shown) to an ultrasound transceiver arm 218 that houses the ultrasound transducer 28. A portion of the transducer and communication cable 240 is seen. The transducer cabling may be connected with the portable console 260 depicted in FIG. 41 below and supported with a torque strain relief suspension 350 depicted in FIG. 44. The transducer/communications cable 240 is generally routed along the user's arm to for coupling to a portable console 260 that is housed in a roller cart 450 depicted in FIGS. 42 and 43. Also included in the blood vessel access device 200 is a pre-sterilized and disposable injector pack 250 that can snap fit into the injector arm 214 which houses the injector 50 described in FIG. 17 above. Inside the injector pack 250 is the needle 20 to which is overlapped a cannula or catheter 252. The needle 20 and cannula 252 are engageable with the needle motion slider 52 and a cannula/catheter motion slider 56. Additional injector packs 250 may be fitted with the injector arm 218 as needed to establish as many access procedures required for a particular patient. In other embodiments, the device 200 ultrasound transducer 28 may be comprised of 25 mm wide linear array elements.

Residing on the injector arm 214 are motion controls 245-248. Control 245 moves the needle 20. Control 246 is a rocker style switch and is configured to send signals that move both the needle 20 and the catheter/cannula 21 located in the cartridge 250. A forward rocking switch movement may send motion signals to move the needle 20 and a rearward rocking switch movement may send motion signals to move the catheter/cannula 21. Control 247 may be configured to move the catheter/cannula 21. Control 248 may be configured to change the orientation of the transducer 28 from short axis to long axis to get a cross-sectional or a longitudinal cross-sectional view of a blood vessel. Control 248 location within the injector arm 214 provides the user to better steady the transducer while in use. The short axis view identifies the anatomy at the start of a blood vessel access procedure, and the long axis view for advancing the needle 20 to penetrate the proximal side of a blood vessel. The control 248 can then be engaged to return to the short axis view to verify alignment of the tip of the needle 20 with a given blood vessel. Thereafter, control 248 may be reengaged to switch to the long axis view to advance the catheter/cannula and withdraw the needle 20. Such configuration allows for 1 and 2 handed operation of device 200, with 1 hand on the transceiver arm 218 and 1 hand on the injector arm 214. Once access of the blood vessel BV is achieved, the user may remove the hand on the injector arm. This 1 or 2 hand operation is sometimes referred to as 1.5 hand operation.

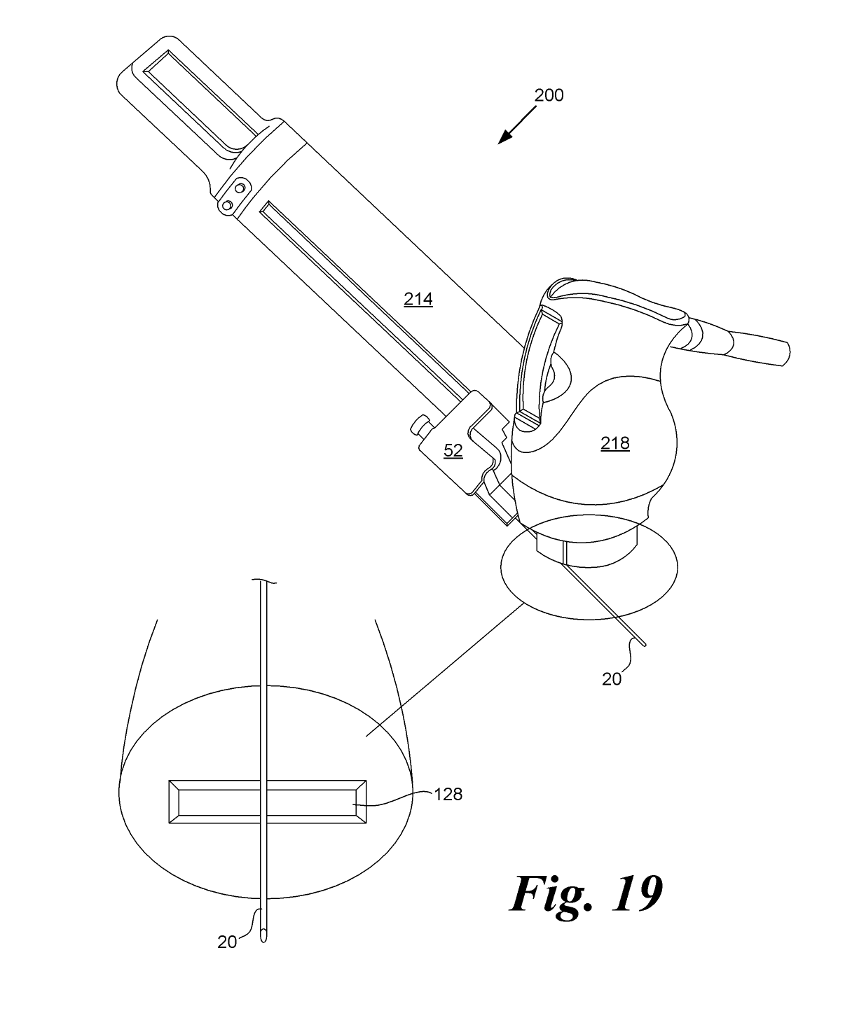

FIG. 19 schematically depicts in greater detail the blood vessel access device 200 operation of the needle motion slider 52 in its advancement of the needle 20 into the viewing range of the transducer 128.

FIG. 20 schematically depicts in greater detail the blood vessel access device 200 arrangement of the display screen 124 in relation to the ultrasound transceiver arm 218. In this case the display screen 124 is fitted with by icon position controls that are touch sensitive and built into the display screen 124. The touch sensitive icon controls include down 230, left 232, right 234, and up 236 to impart image positioning. The in-screen built in touch sensitive icon controls allow for miniaturization of the transducer arm 218 so that it can be more readily stabilized with a user's single hand.

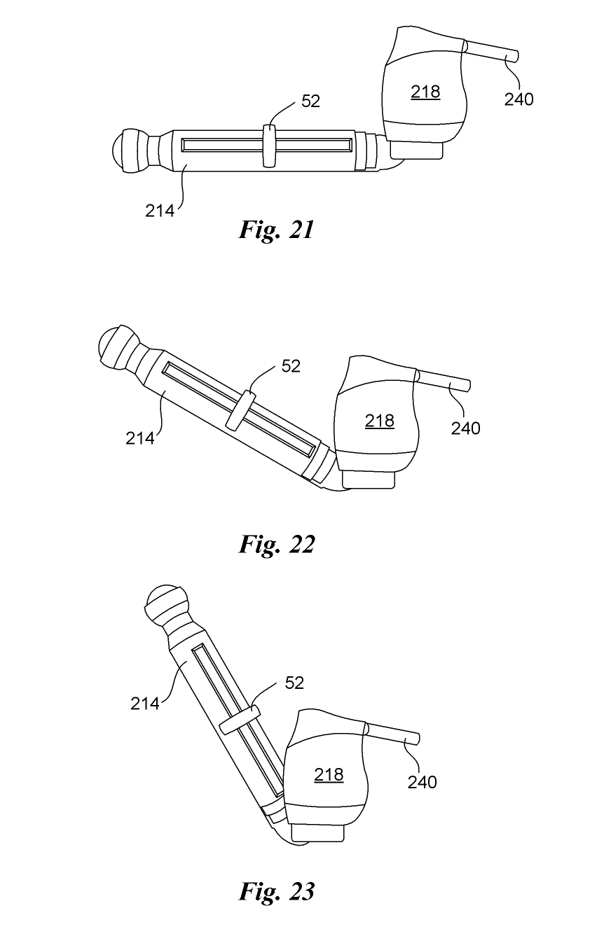

FIG. 21 schematically depicts in side view the injector arm 214 presented at level or zero degrees for loading the disposable injector pack 250.

FIG. 22 schematically depicts in side view the injector arm 214 presented at a shallow and to medium angle for advancing the needle 20 from the injector pack 250 into the patient.

FIG. 23 schematically depicts in side view the injector arm 214 presented at a steep angle for advancing the needle 20 from the injector pack 250 into the patient.

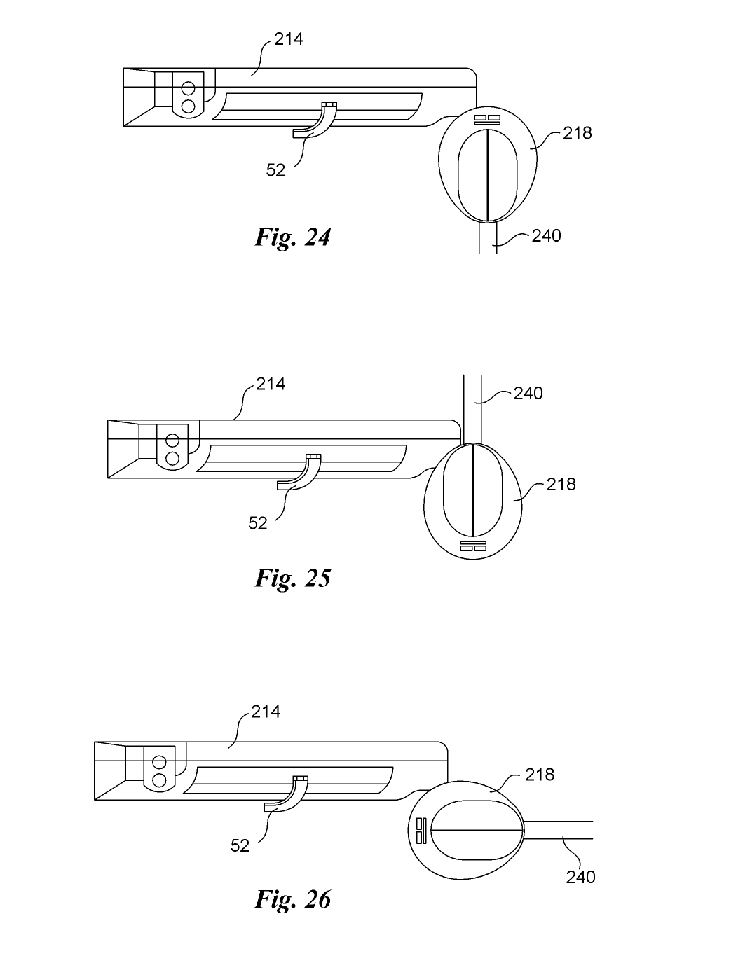

FIG. 24 schematically depicts in top view the transceiver 218 directed to the right side of the patient.

FIG. 25 schematically depicts in top view the transceiver 218 directed to the left side of the patient.

FIG. 26 schematically depicts in top view the transceiver 218 directed straight to the patient.

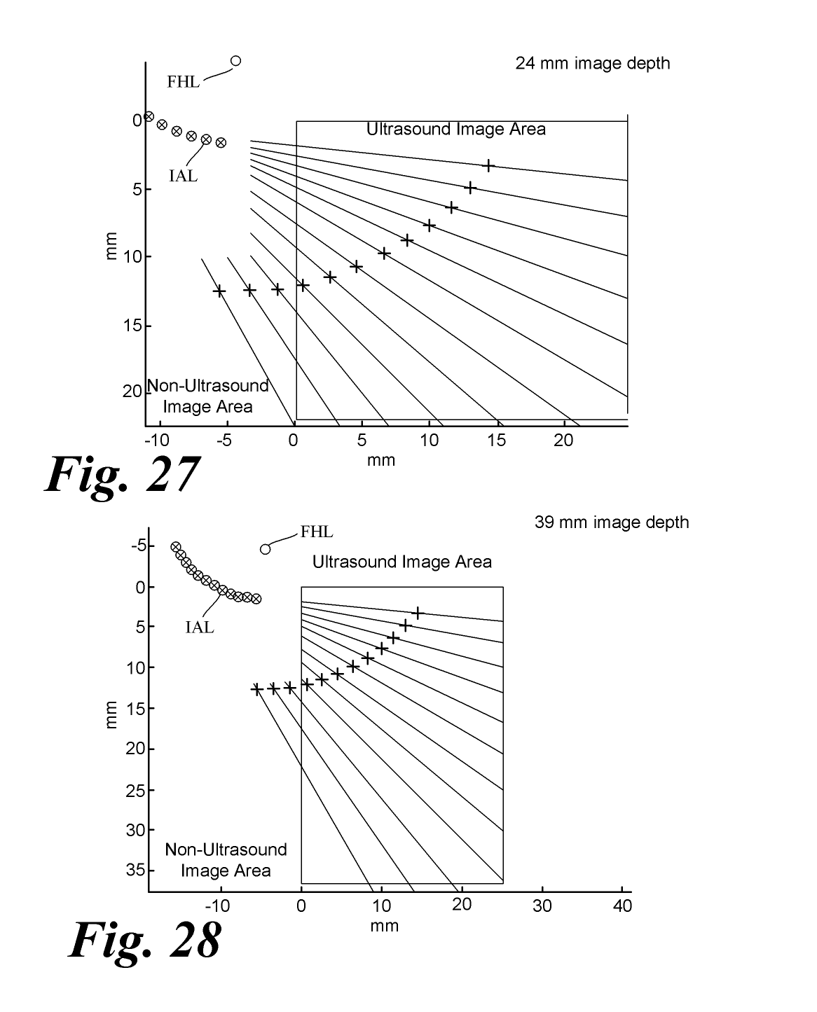

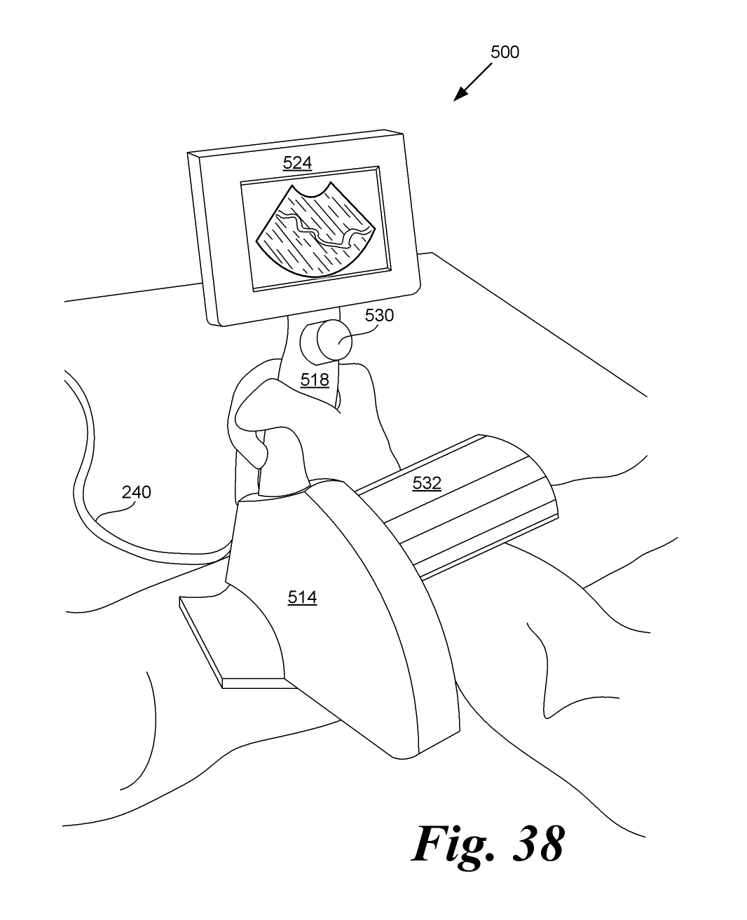

FIG. 27 schematically depicts a plot of exemplary predicted paths entry slopes or needle trajectories required to reach a blood vessel located at 24 mm depth beneath the skin of a patient. The predicted path trajectories are the solid lines the needle 20 can transit based upon the angular increments designated as injector arm locus IAL as the injector arm pivots about the friction hinge designated as friction hinge locus on the 24 mm depth plot. The solid lines traverse through a non-ultrasound image area and the ultrasound image area viewable on the touch-sensitive monitor screens 124 or other screen designated in FIGS. 38-40.

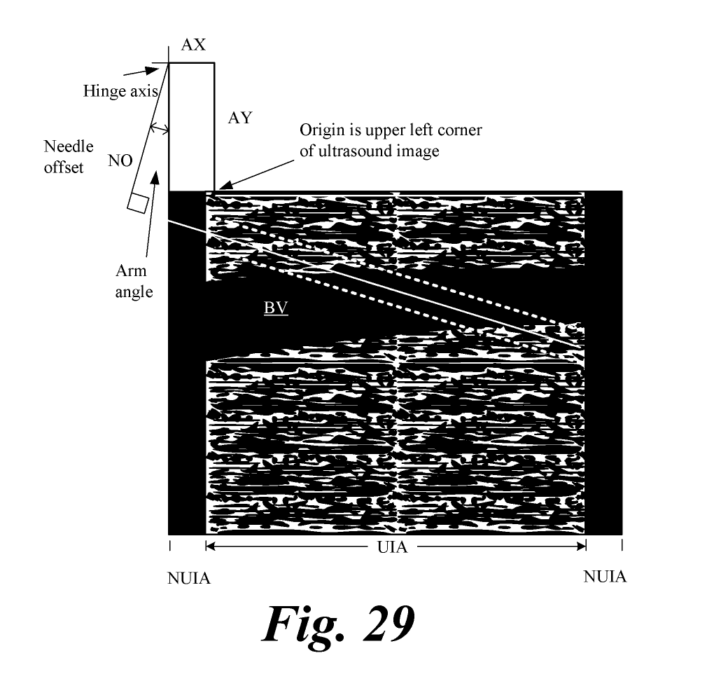

FIG. 28 schematically depicts a plot of predicted paths entry slopes or needle trajectories required to reach a blood vessel located at 39 mm depth beneath the skin of a patient. The predicted path trajectories are the solid lines the needle 20 can transit based upon the angular increments designated as injector arm locus IAL as the injector arm pivots about the friction hinge designated as friction hinge locus on the 39 mm depth plot. The solid lines traverse through a non-ultrasound image area and the ultrasound image area viewable on the touch-sensitive monitor screens 124 or other screen designated in FIGS. 38-40.

FIG. 28 schematically depicts a plot of predicted needle paths entry into a blood vessel BV displayed in longitudinal cross section in the ultrasound image area. The upper left hand corner of the ultrasound image area designates the origin of the ultrasound image. The hinge axis locus (arrow) is described in terms AX and AY. AX designates the horizontal offset of the hinge axis from the ultrasound image from the upper left corner and AY designates the vertical offset of the hinge axis from the ultrasound image upper left corner. Another term designated is NO which is the needle path parallel offset from the path P going through the hinge. The dashed lines represent the predicted path entry of the needle, each dashed line representing opposing surfaces of the needle. The solid line represents the middle of the needle between the two opposing surfaces of the needle designated the dashed lines. Both dashed lines and solid line transit through the non-ultrasound image areas NUIA, the black vertical bars, and middle region which are the ultrasound image area UTA to which the NUIA black bars flank. As seen in this depiction, the passage location of the entry into the proximal side of the blood vessel BV and its exit from the distal side of the blood vessel BV can be ascertained. In this example, AX=−4.5 mm, AY=−11.25 mm, and NO=12.75 MM.

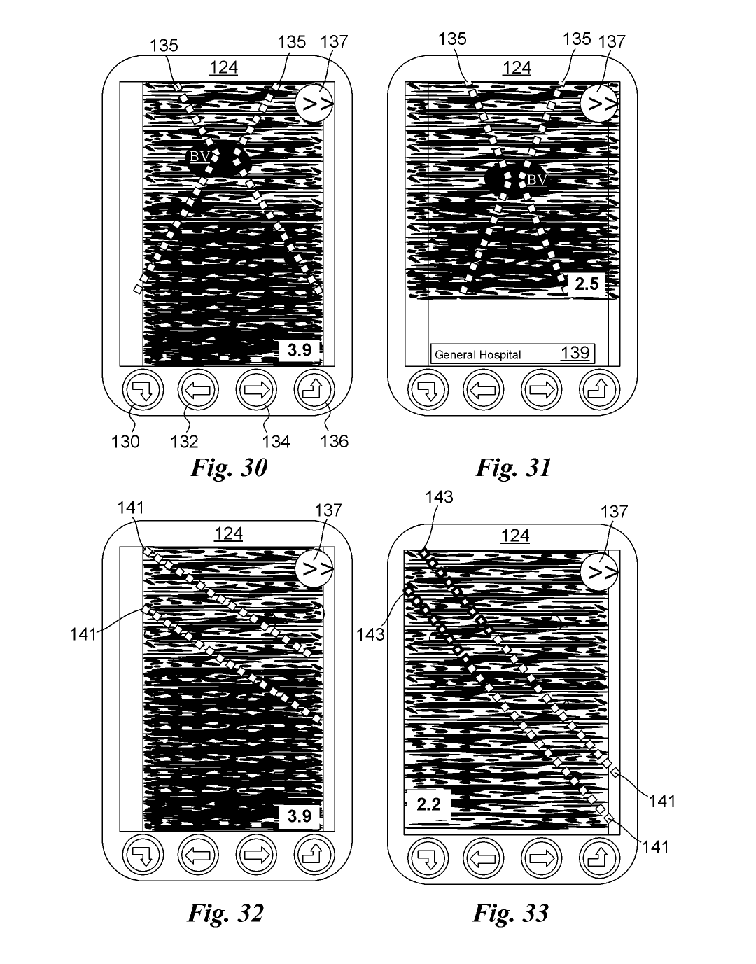

FIGS. 30-36 depict different ultrasound image area presentations on the touch sensitive monitor 124 positioned by engagement of image position buttons 130, 132, 134, and 136.

FIG. 30 illustrates opposing cross hair tracks 135 aimed near the middle of a blood vessel BV located at 3.9 cm depth from transducer 128. Motion icon 137 is with symbol similar to the symbol displayed for either the needle positioning mode 112 or catheter/cannula positioning mode 170 as described in FIG. 7.

FIG. 31 illustrates opposing cross hair tracks 135 aimed near the middle of a blood vessel BV located at 2.5 cm depth from transducer 128. Motion icon 137 is with symbol similar to the symbol displayed for either the needle positioning mode 112 or catheter/cannula positioning mode 170 as described in FIG. 7. Data window 139 allows user to voice annotate the screen image with procedural or technical alphanumeric information.

FIG. 32 illustrates opposing parallel running needle 20 predicted pathways 141, where each pathway 141 represents opposing sides of the needle were it to coursing through a patient's ultrasonically visualized vasculature.

FIG. 33 illustrates opposing parallel running needle 20 predicted pathways 141, where each pathway 141 represents opposing sides of the needle were it to coursing through a patient's ultrasonically visualized vasculature, overlaid with actual image track 143, here presented in boldface, to signify the real presence of the needle 20 coursing through the patient's vasculature.

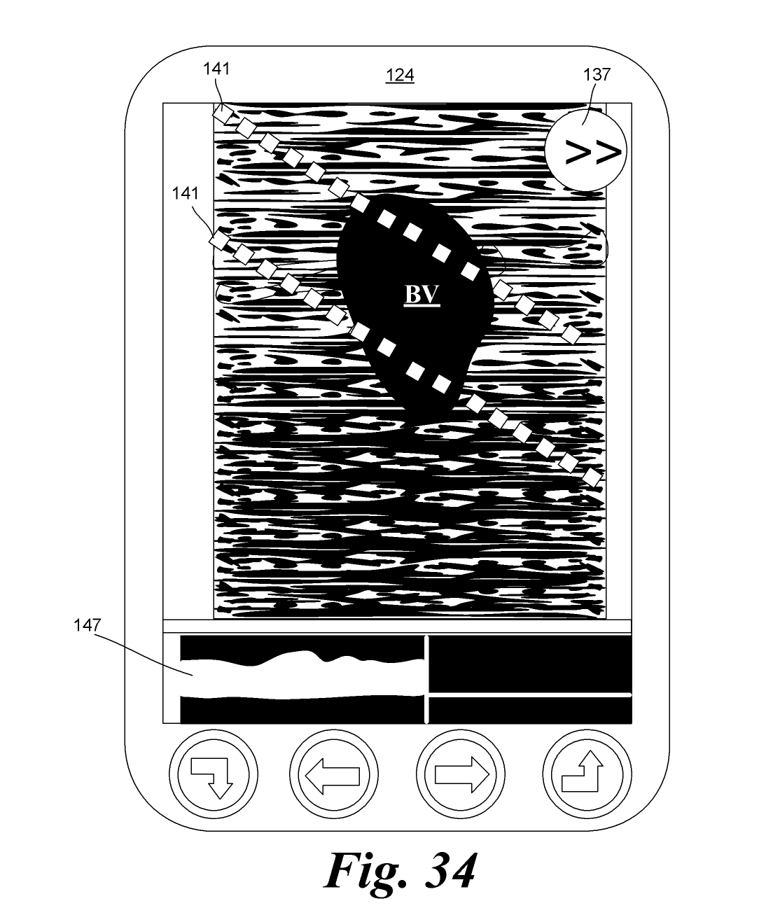

FIG. 34 schematically illustrates an ultrasound image have a lateral cross-sectional image with a predicted pathway 141 coursing through a blood vessel BV. In this ultrasound image, a sonic window 147 indicates a constant audio profile indentifying the blood vessel BV to be a vein.

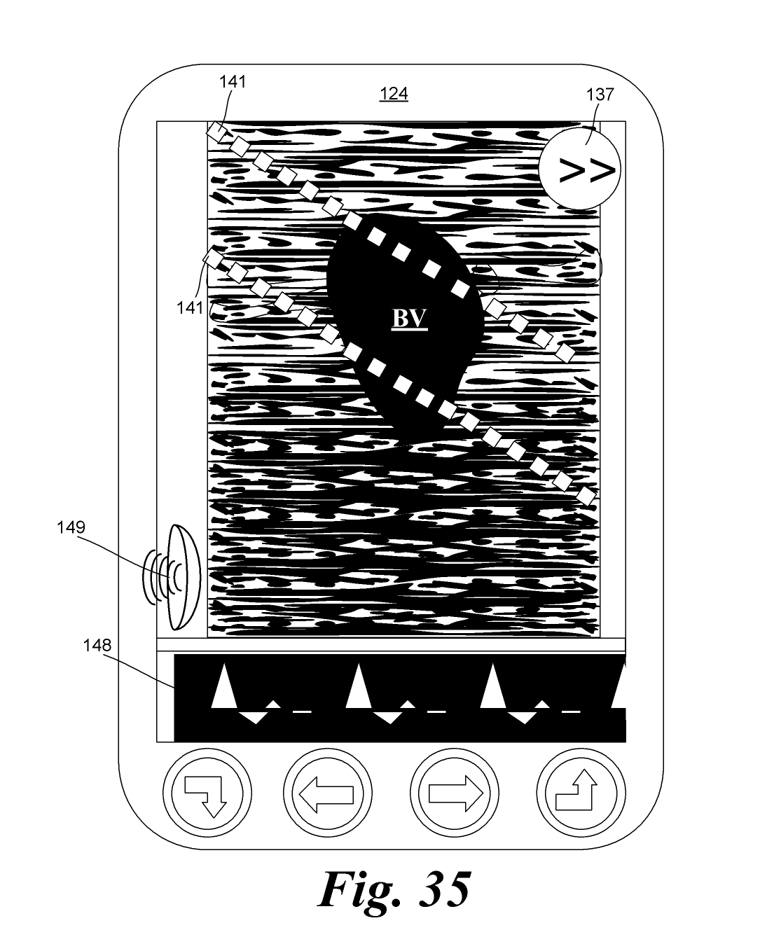

FIG. 35 schematically illustrates an ultrasound image having a lateral cross-sectional image with a predicted pathway 141 coursing through a blood vessel BV. In this ultrasound image, a sonic window 148 indicates a constant audio profile indentifying the blood vessel BV to be an artery. The sonic window 148 may display color Doppler ultrasound to more clearly display the pulsating arterial blood flows. A speaker icon 149 can be displayed with an audio output of the arterial blood flows.

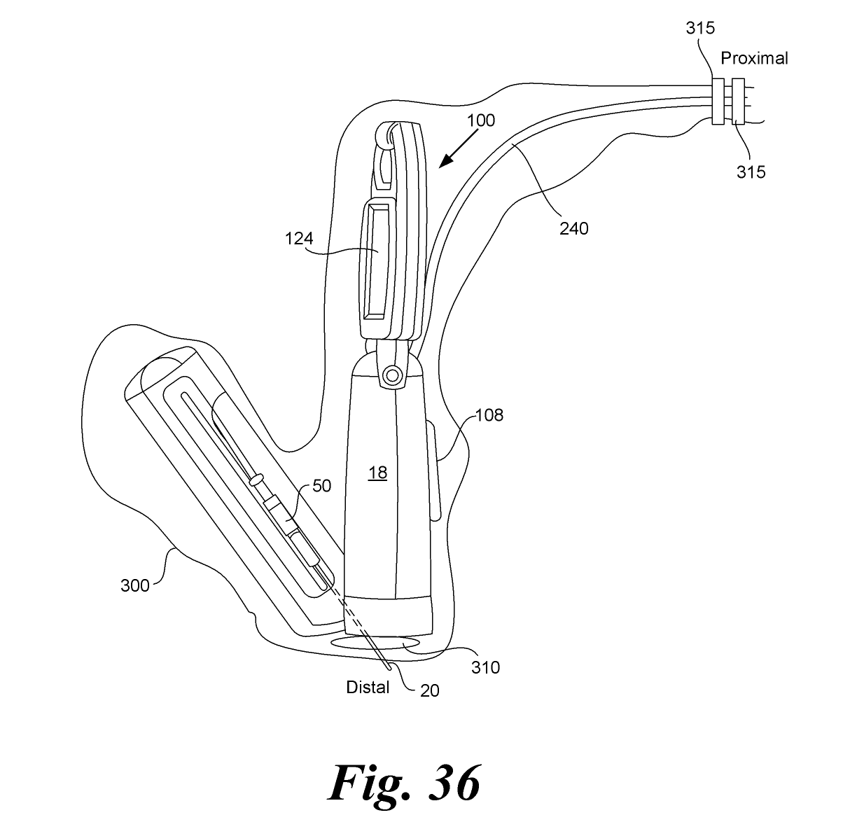

FIG. 36 schematically depicts a sterile sheath 300 having a sonic gel pack 310 enveloping the blood vessel access device 100 to provide ultrasound image guide blood vessel access procedures under sterile conditions. Sterile conditions are desired for the placement of PICC and central lines. The breakable sonic gel pad 310 resides near the transducer 28 at the distal end of the ultrasound transceiver arm 18. The sheath extends and envelops over the proximal sides of the cable 240. The sheath may be tightly secured by to the cable 240 with constricting bands 315. With sterile gloved hands, the user operates the controls through the sheath 300 and the needle 20 punctures the sheath 300 during needle advancement procedures. The sheath 300 is transparent to allow the user to view unhindered images appearing on the monitor screen 124 to permit ultrasound image guided access and cannulation of blood vessels. In alternate embodiments, the cable 240 is replaced by a wireless link similar to the wireless communication 640 Depicted in FIG. 42 to allow the user to conduct image guided access and cannulation via another monitor in view of the user. The sterile sheath 300 drapes over the monitor 124 and readily allows touch screen manipulations by the sterile gloved user.

Additional properties of the sheath 30 is that it includes slack regions to allow motion transmission of the injector arm 14/214 and operation of the control panel 108 so that the sheath 30 does not become torn during access and placement operations except for the puncture site in the sheath 30 while needle 20 advancement and withdrawal occurs. The sheath 30 may be packaged as a sterile entity or within a sterile but tearable pouch configured for ready removal and draping over the blood access devices.

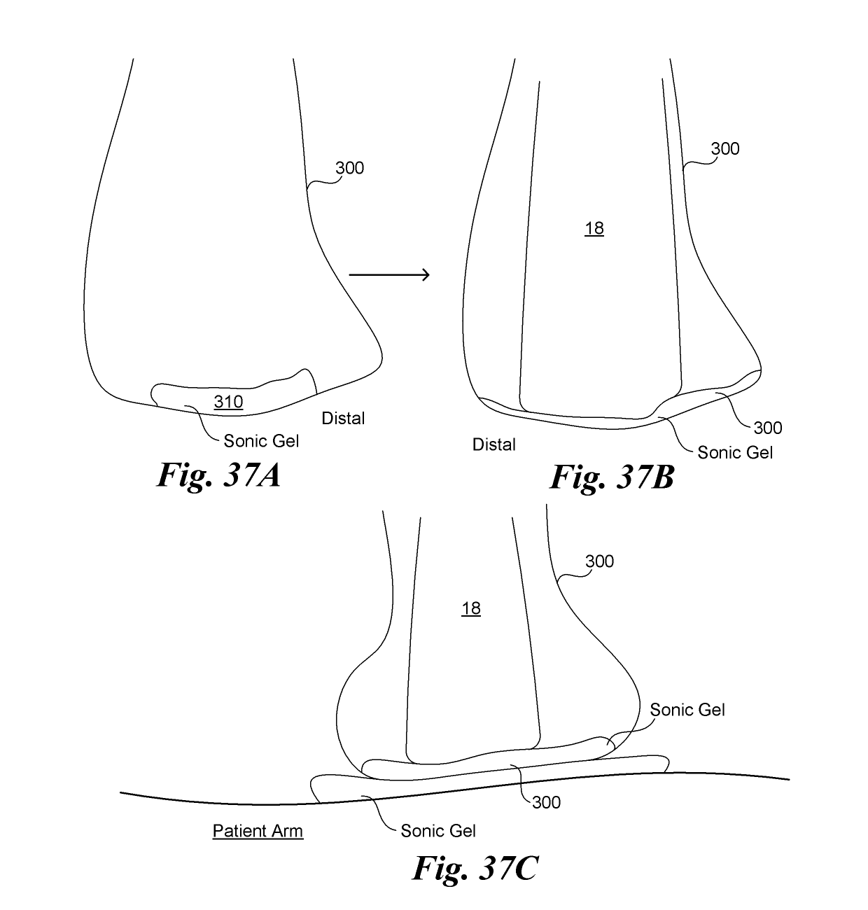

FIGS. 37A-C illustrates aseptic operation of the sterile sheath 300 covering and operation procedure. In FIG. 37A, the sonic gel is spread out at the distal end of the flexible sheath 300. With sterile glove handing, the exterior surface of the sheath 300 is maintained sterile, and sonic gel is spread out internally within the distal end of the sheath 300 by externally squeezing the gel pack 310 to have the sonic coupling gel ooze from the gel pack 310. In FIG. 37B, transceiver arm 18 is brought to bear against the sonic gel and it spreads out to make acoustic connection between the sonic gel and the interior surface of the sheath 300. In FIG. 37C, a sterile application of sonic coupling gel is applied to the patient's arm and the exterior and sterile surface of the flexible sheath 300 is brought to bear against the sonic gel in contact with the patient's arm. In so doing, sonic coupling is established and maintained from the transducer 18 (not shown) and the patient's skin.

FIG. 38 schematically depicts a blood access device 500 configured with an ultrasound transceiver handle 518 that is pivotally connected with the injector 50 (not shown) that resides and pivots within an injector cradle 514. The injector cradle 514 includes friction hinges (not shown) to allow incremental and continuous angle adjustments for needle penetration and catheter placements. The user utilizes a toggle motion control 530 that provides both button pressing and four-way toggling to effect needle penetration and withdrawal and catheter placement and withdrawal in a targeted blood vessel according to the ultrasound images presented on the monitor 525. Transducer and communication cable 240 connects with the console 260 described in FIG. 41. An arm rest 532 secures the user arm and restrains user motion to keep the extraneous motion to a minimum.

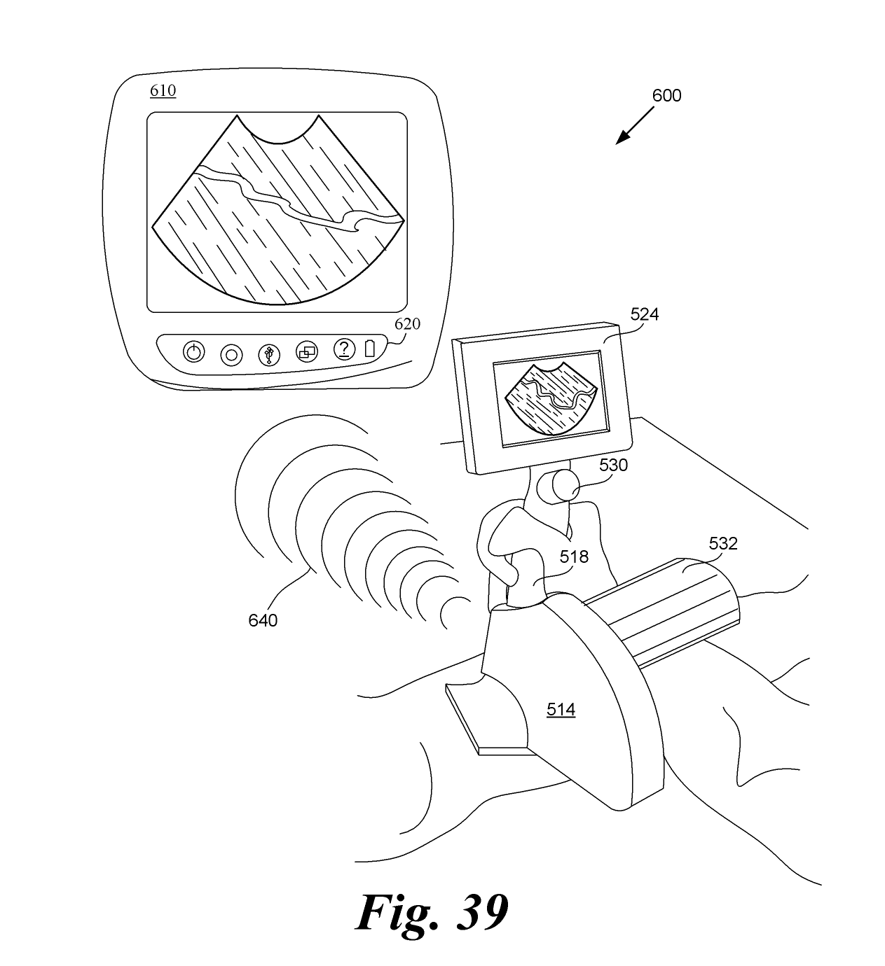

FIG. 39 schematically depicts a blood access device 600 configured for wireless signal 640 communication with another monitor 610. Other components are similar to that described for blood vessel access device 500. The same or different ultrasound based images may be presented on the monitor 524 and the other monitor 610 to provide the same or different views to assist the user executing ultrasound image-guided needle penetration and cannula placement procedures. Image adjustments within the monitor 610 are provided by image control panel 620.

FIG. 40 schematically depicts a blood access device 700 configured for wireless signal 640 communication with the monitor 610. In this embodiment the blood access device 700 does not have an integral monitor similar to the monitor 524 depicted in FIGS. 38 and 39. Console functions described for portable console 260 in FIGS. 16 and 44 may be shared between the transceiver handle 518 and internally within monitor 610.

FIG. 41 schematically depicts a portable console 260. The portable console 260 includes a rechargeable and insertable power supply 270. Console 260 receives the power and communication cable 240 and includes the functional electronics configured to provide microprocessor executable instructions to generate the video processing, ultrasound image generation and storage, and A-mode, B-mode, and C-mode image processing.

FIG. 42 illustrates a roller cart 450 that houses the portable console 260 and transceiver 218.

FIG. 43 illustrates the roller cart 450 with the portable console 260, transceiver 218, and power supply 270 elevated to showing the housing compartments of the roller cart 450.

FIG. 44 illustrates components of the torque strain relief suspension 350 designed to provide support to cable 240 yes not restrict motion of or induce motion to the user movement of the blood vessel access device 200 depicted in FIGS. 18-26. Torsion springs 360 occupy each end of the relief suspension 350 and are paired off in clockwise-counter clockwise fashion. A flex connector 365 is coupled to the transceiver handset end and is ribbon cable 362 connected to an ultrasound acoustic stack 370.

The devices and systems thus described may be used for aseptic and sterile access and cannulation procedures. In general for cannulation of arm or leg blood vessels with short IVs, the aseptic procedure begins with the user selecting an arm of the patient to cannulate and positions the patient in a way that both allows the user to place the blood access devices thus described on the upper arm or other chosen target and maintain comfort to the patient. The user applies ultrasound gel to the cannulation site and obtains an ultrasound image to examine the arm or leg vasculature in cross-section. The user selects a vein for cannulation and presses the selected vein to confirm that its lumen collapses to verify it is a vein and not an artery. Confirmation that it is a vein can be secured by examining the sonic pattern as described for FIGS. 34 and 35. If a thrombose is suspected, another blood vessel target is selected. The cannulation site is then prepped for needle penetration by wiping the cannulation site with antiseptic swabs. A sterile needle-and-cannula cartridge 250 is snapped into place, and the cannulation device is overlaid on the patient's anatomy having the antiseptic wiped cannulation site. Ultrasound images are procured and the user examines blood vessel candidates for cannulation in cross-section. Aiming crosshairs are placed on the top wall of the targeted blood vessel. The user then switches to longitudinal cross-sectional view and operates the motion control buttons in control panel 108 to advance the needle 20 into the blood vessel. Thereafter, the user switches to cross-section view and verifies the needle 20 tip is immediately on tip of the vessel. The user moves the blood vessel access devices previously described to correct for any deviation. When the needle 20 tip is in the center of the blood vessel, the user advances the catheter or cannula into the blood vessel. Once the catheter is fully advanced, the user withdraws the needle 20 back into the cartridge 250, thereby preventing accidental sticking the user.

For sterile operations, as is commonly desired for placement of longer PICC lines, the procedure is similar to the aseptic procedure except that a sterile field is set up around the selected arm or leg. At this point, the user may either envelop the blood vessel access device with a sterile sheath 300 having a breakable sonic gel pad as described for FIG. 36-37C using freshly adorned sterile gloves, or apply sonic gel directly the ultrasound transducer 28 and then place inside a sterile sheath using freshly adorned sterile gloves. Upon placing the sheath enveloped blood vessel access device over the aseptically prepared cannulation site within the sterile field with freshly adorned sterile gloves, blood vessel targeting and cannulation procedures are executed for the aseptic procedure, except that the user frequently replaces and covers his hands with freshly adorned sterile gloves.

While the preferred embodiment of the invention has been illustrated and described, as noted above, many changes can be made without departing from the spirit and scope of the invention. For example, in alternate embodiments the display screens 124 may include the sections to display voice recorded alphanumeric messages during blood vessel access procedures. Access procedure progress bars may also be overlaid on the real time ultrasound images. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. An access system operated by a user directed to placing a needle in a blood vessel of a patient, the system comprising:
an ultrasound transceiver sonically coupled to convey ultrasound energy into the patient and generate signals from the ultrasound energy returning from the patient;
a monitor configured to present an image having at least one blood vessel derived from the signals;
a needle injector pivotally connected with the ultrasound transceiver, the needle injector having a needle configured for slidable connection with a cannula; and
a controller in communication with the needle injector;
wherein positioning of the needle within the blood vessel is determined by operation of the controller by the user viewing the image.

2. The access system of claim 1, wherein the image includes at least one of a cross-sectional view, a longitudinal cross-sectional view, and a three dimensional rendering of the at least one blood vessel.

3. The access system of claim 1, wherein the image includes a window to display a sonic pattern to discern whether the at least one blood vessel is an artery or a vein.

4. The access system of claim 1, wherein the image includes a window to display color Doppler graphics to discern whether the at least one blood vessel is an artery or a vein.

5. The access system of claim 1, wherein the needle injector is includes a cannula that at least partially overlaps the needle.

6. The access system of claim 1, wherein the needle injector is includes a cannula that at least partially overlaps the needle and is contained within a sterilizable housing detachably connectable with the needle injector.

7. The access system of claim 1, wherein the ultrasound transceiver, the needle injector, and the controller is configured for enveloping within a flexible sheathing capable of undergoing a sterilization process.

8. The access system of claim 1, wherein the image includes an overlay presenting a predicted path of the needle from the needle injector to the lumen of the at least one blood vessel.

9. The access system of claim 1, wherein the image includes an overlay presenting a predicted path of the needle from the needle injector to the lumen of the at least one blood vessel, the overlay presented on the monitor displaying at least one of a transverse cross-sectional view, a longitudinal cross-sectional view, and a three dimensional view of the at least one blood vessel.

10. An access device operated by a user in conjunction with an ultrasound transceiver having a monitor configured to display an image of at least one blood vessel of a patient, the device comprising:
a needle injector pivotally connected with the ultrasound transceiver, the needle injector having a needle configured for slidable connection with a cannula; and
a controller in communication with the needle injector;
wherein positioning of the needle within the blood vessel is determined by operation of the controller by the user viewing the image.

11. The access device of claim 10, wherein the image includes at least one of a transverse cross-sectional view, a longitudinal cross-sectional view, and a three dimensional rendering of the at least one blood vessel.

12. The access device of claim 10, wherein the needle injector is includes a cannula that at least partially overlaps the needle.

13. The access device of claim 10, wherein the needle injector is includes a cannula that at least partially overlaps the needle and is contained within a sterilizable housing detachably connectable with the needle injector.

14. The access device of claim 10, wherein the ultrasound transceiver, the needle injector, and the controller is configured for enveloping within a flexible sheathing capable of undergoing a sterilization process.

15. The access device of claim 10, wherein the image includes an overlay presenting a predicted path of the needle from the needle injector to the lumen of the at least one blood vessel.

16. The access device of claim 10, wherein the image includes an overlay presenting a predicted path of the needle from the needle injector to the lumen of the at least one blood vessel, the overlay presented on the monitor displaying at least one of a transverse cross-sectional view, a longitudinal cross-sectional view, and a three dimensional view of the at least one blood vessel.

17. A method to access at least one blood vessel of a patient, the method comprising:
connecting a needle injector pivotally with an ultrasound transceiver having a monitor configured to present an image of the at least one blood vessel, the needle injector having a needle configured for slidable connection with a cannula;
overlaying a predicted path of the needle for entry into the at least one blood vessel;
operating a controller in communication with the needle injector;
positioning the needle within the blood vessel via operation of the controller by the user viewing the predicted path undertaking by the needle as appearing within the image.

18. The access method of claim 17, wherein the overlay is presented on the monitor displaying at least one of a transverse cross-sectional view, a longitudinal cross-sectional view, and a three dimensional view of the at least one blood vessel.

19. A method to access at least one blood vessel of a patient, the method comprising:
connecting a needle injector pivotally with an ultrasound transceiver having a monitor configured to present an image of the at least one blood vessel, the needle injector connectable with a sterilizable housing having a needle configured for slidable connection with a cannula;
overlaying a predicted path of the needle for entry into the at least one blood vessel;
operating a controller in communication with the needle injector;
positioning the needle within the blood vessel via operation of the controller by the user viewing the predicted path undertaking by the needle as appearing within the image.

20. The access method of claim 20, wherein the overlay is presented on the monitor displaying at least one of a transverse cross-sectional view, a longitudinal cross-sectional view, and a three dimensional view of the at least one blood vessel.

21. A method to access at least one blood vessel of a patient, the method comprising:
connecting a needle injector occupying pivotally with an ultrasound transceiver having a monitor configured to present an image of the at least one blood vessel, the needle injector connectable with a sterilizable housing having a needle configured for slidable connection with a cannula;
enveloping the needle injector, the ultrasound transceiver, and the sterilizable housing within a flexible, sterilizable sheath;
overlaying a predicted path of the needle for entry into the at least one blood vessel;
operating a controller in communication with the needle injector;
positioning the needle within the blood vessel via operation of the controller by the user viewing the predicted path undertaken by the needle as appearing within the image.

22. The access method of claim 20, wherein the overlay is presented on the monitor displaying at least one of a transverse cross-sectional view, a longitudinal cross-sectional view, and a three dimensional view of the at least one blood vessel.
